(19) United States
(12) Patent Application Publication
(10) Pub. No.: US 2025/0283129 A1
(43) Pub. Date: Sep. 11, 2025

(54) PROCESS

(71) Applicant: Sun Pharmaceutical Industries Australia Pty Limited, Notting Hill (AU)

(72) Inventors: Xu Li, Melbourne (AU); Sally Louise Gras, Melbourne (AU); Garrick Westley Kyle Spencer, Melbourne (AU)

(73) Assignee: Sun Pharmaceutical Industries Australia Pty Limited, Notting Hill (AU)

(21) Appl. No.: 18/276,606

(22) PCT Filed: Feb. 9, 2022
(86) PCT No.: PCT/AU2022/050077

§ 371 Date: Aug. 9, 2023

(30) Foreign Application Priority Data
Feb. 10, 2021 (GB) 2101796.7

(51) Int. Cl. C12P 17/18; C12N 9/02; C12N 15/82

(52) U.S. Cl. CPC C12P 17/18; C12N 9/0071; C12N 15/8243; C12Y 114/11032

(57) ABSTRACT

The disclosure relates to a biotransformation for the manufacture of one or more opiate or opioid alkaloids using a crude poppy extract or a purified or semi-purified source of opiate or opioid alkaloid; vectors comprising nucleic acid molecules encoding enzymes; microbial cells expressing nucleic acids encoding said enzymes and including modified enzymes with enhanced activity.

### CROSS REFERENCE TO RELATED APPLICATIONS

This is the § 371 U.S. National Stage of International Application No. PCT/AU2022/050077, filed Feb. 9, 2022, which was published in English under PCT Article 21 (2), which in turn claims the benefit of GB Application No. 2101796.7, filed Feb. 10, 2021, which is incorporated herein by reference in its entirety.

### FIELD OF THE DISCLOSURE

The disclosure relates to a process for the manufacture of one or more opiate or opioid alkaloids wherein the process comprises the formation of a reaction mixture including a host cell transformed with one or more nucleic acid molecules encoding an enzyme catalysing at least one reaction in the conversion of an opiate alkaloid, including semi-synthetic opioid alkaloids, and reacting the mixture to convert said opiate alkaloid, or semi-synthetic opioid alkaloid, into a further opiate or opioid alkaloid and optionally extracting the product(s) of the reaction mixture; vectors comprising said nucleic acid molecules; and host cells expressing nucleic acids encoding polypeptides and modified polypeptides with altered or enhanced activity.

### SEQUENCE LISTING

The Sequence Listing is submitted as an ASCII text file in the form of the file named 8617-110547-01_ST25.txt, which was created on Feb. 2, 2024, and is 1,007,988 bytes, which is incorporated by reference herein.

### BACKGROUND TO THE DISCLOSURE

Opiates such as codeine and morphine are potent analgesics and are frequently used to treat moderate to strong pain. Semi-synthetic opioids such as oxycodone, oxymorphone, nalbuphine, naloxone, naltrexone, buprenorphine or etorphine have often a lower side effect profile when compared to codeine and morphine thus offering a valuable pain-management-alternative.

The biosynthetic pathway of morphinan alkaloids is well established and includes a series of enzymatic steps starting from dopamine and 4-hydroxyphenylacetaldehyde leading to the synthesis of R-reticuline which is subsequently transformed to thebaine. Thebaine is then converted to either oripavine by the codeine 3-O-demethylase (CODM) or transformed in several steps to codeine involving the activity of the thebaine-6-O-demethylase (T6ODM). Codeine is subsequently converted to morphine by de-methylation by CODM.

Thebaine is a starting material for the synthesis of some semi-synthetic opioids. Whilst opiate alkaloids can be extracted from latex, harvested from the green seed pods of opium poppy or from the poppy straw, which is the dried mature plant, the demand for the opiate alkaloids in general exceeds the available natural supply necessitating costly chemical synthesis. Thebaine, the chemical precursor of codeine, morphine and semi-synthetic opioids is readily converted in the plant and thus the level of thebaine in the wild type poppy plant is low.

WO2017/122011, which is incorporated by reference in its entirety, discloses the characterisation of the genomic locus encoding CODM genes in Papaver somniferum. The locus includes three closely linked CODM genes that are around 99% identical at the level of genomic sequence. Mutations in the CODM genes that affect enzyme activity are associated with elevated codeine and in plants carrying a deletion of each of the CODM genes the plants have very high levels of codeine. EP3398430, which is incorporated by reference in its entirety, is disclosed the characterisation of the genomic locus encoding T6ODM genes. The locus includes five T6ODM genes. In P. somniferum plants that carry mutations in both the CODM three gene cluster combined with mutations in T6ODM genes the resulting double mutants show high levels of thebaine. If CODM and T6ODM expression or activity is undetectable the resulting plants have very high levels of thebaine.

We disclose a process for the conversion of one or more opiate or opioid alkaloids to one or more opiate or opioid intermediates comprising forming a preparation comprising a host cell, for example a bacterial, fungal or plant cell, transformed with a one or more nucleic acids encoding an enzyme involved in opiate biotransformation, or an enzyme involved in the synthesis of semi-synthetic opioids such as oxycodone, oxymorphone, nalbuphine, naloxone, naltrexone, buprenorphine or etorphine.

We also disclose the use of a crude poppy extract obtained from a poppy, for example P. sominferum, incubating the preparation to obtain a biotransformation and optionally extracting one or more opiate or opioid alkaloids from the transformed preparation. The disclosure provides a scaled transformation of opiate alkaloids as an alternative to in planta extraction of opiate alkaloids from Papaver species. We also disclose CODM polypeptides carrying amino acid modifications which result in altered or enhanced CODM activity.

### STATEMENTS OF INVENTION

According to an aspect of the invention there is provided a method for the biotransformation of one or more opiate alkaloids or synthetic opioid alkaloids comprising the steps:
forming a preparation comprising a host cell transformed with one or more nucleic acid molecules encoding one or more polypeptides catalysing at least one reaction in the conversion of one or more opiate alkaloids or synthetic opioid alkaloids to one or more different opiate alkaloids or semi-synthetic opioid alkaloids;
incubating the reaction to allow transformation of one or more opiates alkaloids or semi-synthetic opioid alkaloids; and optionally
extracting said one or more transformed different opiate alkaloids or semi-synthetic opioid alkaloids from said preparation.

In a preferred method of the invention said preparation comprises naturally occurring opiate alkaloids.

In an alternative preferred method of the invention said preparation comprises semi-synthetic opioid alkaloids.

In a preferred method of the invention said preparation comprises a crude poppy extract comprising natural opiate alkaloids, for example, codeine, thebaine, codeinone and morphinone.

In a preferred method said preparation comprises an opiate alkaloid is selected from the group consisting of thebaine, oripavine, codeine, morphinone, neomorphinone, neopinone and codeinone, preferably thebaine and codeine.

In a preferred method said preparation comprises codeine or thebaine.

In a preferred method said one or more transformed different opiate alkaloids are selected from the group consisting of oripavine, codeine, morphinone, neomorphinone, neopinone and codeinone, preferably thebaine and codeine.

In a preferred method said one or more transformed different opiate alkaloids is oripavine and morphine.

In an alternative method of the invention said opioid alkaloid is a semi-synthetic opioid alkaloid selected from the group consisting of: oxycodone, oxymorphone, hydrocodone, hydromorphone, dihydrocodeine, dihydromorphine: 14-hydroxycodeine, 14-hydroxymorphine, noroxycodone, noroxymorphone, noroxycodeinone, noroxymorphinone, 14-hydroxy norcodeinone, 14-hydroxy normorphinone, 14-hydroxycodeinone, 14-hydroxymorphinone, buprenorphine intermediates such as for example N-cyclopropylmethyl-7α-(2-hydroxy-3,3-dimethyl-2-butyl)-6,14-endoethano-6,7,8,14-tetrahydronorthebaine or 7-acetyl-6,14-endoetheno-6,7,8,14-Tetrahydrothebaine, buprenorphine ((2S)-2-[17-(cyclopropylmethyl)-4,5α-epoxy-3-hydroxy-6-methoxy-6α,14-ethano-14α-morphinan-7α-γl]-3,3-dimethylbutan-2-ol), and etorphine intermediate.

Further examples of semi-synthetic opioid alkaloids as disclosed in US2019/0144900 which is incorporated by reference in its entirety.

In a preferred method of the invention said nucleic acid molecule comprises a nucleotide sequence selected from the group consisting of:
i) a nucleotide sequence as set forth in SEQ ID NO: 1, 2, 3, 4, 5, 6, 7, 8, 9, 10, 11, 12, 13, 14 or 15;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridization conditions to the sequence in SEQ ID NO: 1, 2, 3, 4, 5, 6, 7, 8, 9, 10, 11, 12, 13, 14 or 15 wherein said nucleic acid molecule encodes a codeine 3-O-demethylase;
iv) a nucleotide sequence that encodes a polypeptide comprising an amino acid sequence as represented in SEQ ID NO: 19, 20, 21, 22, 23, 24, 25, 26, 27, 28, 29, 30 or 31;
v) a nucleotide sequence that encodes a polypeptide comprising an amino acid sequence wherein said amino acid sequence is modified by addition, deletion or substitution of at least one amino acid residue as represented in iv) above and which has codeine 3-O-demethylase activity.

In a preferred embodiment of the invention said nucleotide sequence encoding codeine 3-O-demethylase polypeptide is set forth in SEQ ID NO: 3 or comprising a nucleotide sequence that is at least 90% identical to SEQ ID NO: 3.

Sequence homology is 90%, 91%, 92%, 93%, 94%, 95%, 96%, 97%, 98% or 99% identity or similarity to the full-length nucleotide or amino acid sequence herein disclosed.

In a preferred embodiment of the invention said nucleotide sequence encoding codeine 3-O-demethylase polypeptide is set forth in SEQ ID NO: 8 or comprising a nucleotide sequence that is at least 90% identical to SEQ ID NO: 8.

In a preferred embodiment of the invention said nucleotide sequence encoding codeine 3-O-demethylase polypeptide is selected from the group consisting of SEQ ID NO 1, 2, 4, 5, 6, 7, 9, 10, 11, 12, 13, 14 and 15, or comprising a nucleotide sequence that is at least 90% identical to SEQ ID NO: 1, 2, 4, 5, 6, 7, 9, 10, 11, 12, 13, 14 and 15.

In a preferred embodiment of the invention said codeine 3-O-demethylase polypeptide comprises an amino acid sequence as set forth in SEQ ID NO: 19, or a polypeptide comprising the amino acid sequence that is at least 90% identical to SEQ ID NO: 19.

In a preferred embodiment of the invention said codeine 3-O-demethylase polypeptide comprises an amino acid sequence as set forth in SEQ ID NO: 20, or a polypeptide comprising the amino acid sequence that is at least 90% identical to SEQ ID NO: 20 and includes the amino acid substitution E259K.

In a preferred embodiment of the invention said codeine 3-O-demethylase polypeptide comprises an amino acid sequence as set forth in SEQ ID NO: 21, or a polypeptide comprising the amino acid sequence that is at least 90% identical to SEQ ID NO: 21 and includes the amino acid substitution E259D.

In a preferred embodiment of the invention said codeine 3-O-demethylase polypeptide comprises an amino acid sequence as set forth in SEQ ID NO: 22, or a polypeptide comprising the amino acid sequence that is at least 90% identical to SEQ ID NO: 22 and includes the amino acid substitution E259H.

In a preferred embodiment of the invention said codeine 3-O-demethylase polypeptide comprises an amino acid sequence as set forth in SEQ ID NO: 23, or a polypeptide comprising the amino acid sequence that is at least 90% identical to SEQ ID NO: 23 and includes the amino acid substitution E259Q.

In a preferred embodiment of the invention said codeine 3-O-demethylase polypeptide comprises an amino acid sequence as set forth in SEQ ID NO: 24, or a polypeptide comprising the amino acid sequence that is at least 90% identical to SEQ ID NO: 24 and includes the amino acid substitution E259A.

In a preferred embodiment of the invention said codeine 3-O-demethylase polypeptide comprises an amino acid sequence as set forth in SEQ ID NO: 25, or a polypeptide comprising the amino acid sequence that is at least 90% identical to SEQ ID NO: 25 and includes the amino acid substitution E259S.

In a preferred embodiment of the invention said codeine 3-O-demethylase polypeptide comprises an amino acid sequence as set forth in SEQ ID NO: 26 or a codeine 3-O-demethylase polypeptide comprising the amino acid sequence that is at least 90% identical to SEQ ID NO: 26 and includes the amino acid substitution E259G.

In a preferred embodiment of the invention said codeine 3-O-demethylase polypeptide comprises an amino acid sequence as set forth in SEQ ID NO: 27, or a polypeptide comprising the amino acid sequence that is at least 90% identical to SEQ ID NO: 27 and includes the amino acid substitution R260T.

In a preferred embodiment of the invention said codeine 3-O-demethylase polypeptide comprises an amino acid sequence as set forth in SEQ ID NO: 28, or a polypeptide comprising the amino acid sequence that is at least 90% identical to SEQ ID NO: 28 and includes the amino acid substitution E259G and R260T.

In a preferred embodiment of the invention said codeine 3-O-demethylase polypeptide comprises an amino acid sequence as set forth in SEQ ID NO: 29, or a polypeptide comprising the amino acid sequence that is at least 90% identical to SEQ ID NO: 29 and includes the amino acid substitution R260K.

In a preferred embodiment of the invention said codeine 3-O-demethylase polypeptide comprises an amino acid sequence as set forth in SEQ ID NO: 30, or a polypeptide comprising the amino acid sequence that is at least 90% identical to SEQ ID NO: 30 and includes the amino acid substitution E259D and R260K.

In a preferred embodiment of the invention said codeine 3-O-demethylase polypeptide comprises an amino acid sequence as set forth in SEQ ID NO: 31, or a polypeptide comprising the amino acid sequence that is at least 90% identical to SEQ ID NO: 31 and includes the amino acid substitution E259G and R260K.

In a preferred method of the invention said nucleic acid molecule comprises a nucleotide sequence selected from the group consisting of:
i) a nucleotide sequence as set forth in SEQ ID NO: 37, 38, 39, 40, 41, 42, 43, 44, 45, 46, 47, 48, 49, 50, 51 or 52;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridization conditions to the sequence in SEQ ID NO: 37, 38, 39, 40, 41, 42, 43, 44, 45, 46, 47, 48, 49, 50, 51 or 52 wherein said nucleic acid molecule encodes a codeine 3-O-demethylase;
iv) a nucleotide sequence that encodes a polypeptide comprising an amino acid sequence as represented in SEQ ID NO: 53, 54, 55, 56, 57, 58, 59, 60, 61, 62, 63, 64, 65, 66, 67 or 68;
v) a nucleotide sequence that encodes a polypeptide comprising an amino acid sequence wherein said amino acid sequence is modified by addition, deletion or substitution of at least one amino acid residue as represented in iv) above and which has codeine 3-O-demethylase activity.

In a preferred embodiment of the invention said nucleotide sequence encoding codeine 3-O-demethylase polypeptide is selected from the group consisting of SEQ ID NO 37, 38, 39, 40, 41, 42, 43, 44, 45, 46, 47, 48, 49, 50, 51 and 52, or comprising a nucleotide sequence that is at least 90% identical to a sequence selected from the group consisting of: SEQ ID NO: 37, 38, 39, 40, 41, 42, 43, 44, 45, 46, 47, 48, 49, 50, 51 and 52.

In a preferred embodiment of the invention said nucleotide sequence encoding a codeine 3-O-demethylase polypeptide is set forth in SEQ ID NO: 47 or comprising a nucleotide sequence that is at least 90% identical to SEQ ID NO: 47.

In a preferred embodiment of the invention said nucleotide sequence encoding a codeine 3-O-demethylase polypeptide is set forth in SEQ ID NO: 43 or comprising a nucleotide sequence that is at least 90% identical to SEQ ID NO: 43.

In a preferred embodiment of the invention said nucleotide sequence encoding a codeine 3-O-demethylase polypeptide is set forth in SEQ ID NO: 52 or comprising a nucleotide sequence that is at least 90% identical to SEQ ID NO: 52.

In a preferred embodiment of the invention said nucleotide sequence encoding a codeine 3-O-demethylase polypeptide is set forth in SEQ ID NO: 51 or comprising a nucleotide sequence that is at least 90% identical to SEQ ID NO: 51.

In a preferred embodiment of the invention said nucleotide sequence encoding a codeine 3-O-demethylase polypeptide is set forth in SEQ ID NO: 50 or comprising a nucleotide sequence that is at least 90% identical to SEQ ID NO: 50.

In a preferred embodiment of the invention said codeine 3-O-demethylase polypeptide comprises an amino acid sequence as set forth in SEQ ID NO: 53, or a polypeptide comprising the amino acid sequence that is at least 90% identical to SEQ ID NO: 53.

In a preferred embodiment of the invention said codeine 3-O-demethylase polypeptide comprises an amino acid sequence as set forth in SEQ ID NO: 54, or a polypeptide comprising the amino acid sequence that is at least 90% identical to SEQ ID NO: 54 and includes the amino acid substitution T3K, P4A, 15K and 17M.

In a preferred embodiment of the invention said codeine 3-O-demethylase polypeptide comprises an amino acid sequence as set forth in SEQ ID NO: 55, or a polypeptide comprising the amino acid sequence that is at least 90% identical to SEQ ID NO: 55 and includes the amino acid substitution Y357S and M360I.

In a preferred embodiment of the invention said codeine 3-O-demethylase polypeptide comprises an amino acid sequence as set forth in SEQ ID NO: 56, or a polypeptide comprising the amino acid sequence that is at least 90% identical to SEQ ID NO: 56 and includes the amino acid substitution T3K, P4A, 15K, 17M, Y357S and M360I.

In a preferred embodiment of the invention said codeine 3-O-demethylase polypeptide comprises an amino acid sequence as set forth in SEQ ID NO: 57, or a polypeptide comprising the amino acid sequence that is at least 90% identical to SEQ ID NO: 57 and includes the amino acid substitution T3K.

In a preferred embodiment of the invention said codeine 3-O-demethylase polypeptide comprises an amino acid sequence as set forth in SEQ ID NO: 58, or a polypeptide comprising the amino acid sequence that is at least 90% identical to SEQ ID NO: 58 and includes the amino acid substitution P4A.

In a preferred embodiment of the invention said codeine 3-O-demethylase polypeptide comprises an amino acid sequence as set forth in SEQ ID NO: 59, or a polypeptide comprising the amino acid sequence that is at least 90% identical to SEQ ID NO: 59 and includes the amino acid substitution 15K.

In a preferred embodiment of the invention said codeine 3-O-demethylase polypeptide comprises an amino acid sequence as set forth in SEQ ID NO: 60 or a codeine 3-O-demethylase polypeptide comprising the amino acid sequence that is at least 90% identical to SEQ ID NO: 60 and includes the amino acid substitution 17M.

In a preferred embodiment of the invention said codeine 3-O-demethylase polypeptide comprises an amino acid sequence as set forth in SEQ ID NO: 61, or a polypeptide comprising the amino acid sequence that is at least 90% identical to SEQ ID NO: 61 and includes the amino acid substitution Y357S.

In a preferred embodiment of the invention said codeine 3-O-demethylase polypeptide comprises an amino acid sequence as set forth in SEQ ID NO: 62, or a polypeptide comprising the amino acid sequence that is at least 90% identical to SEQ ID NO: 62 and includes the amino acid substitution M360I.

In a preferred embodiment of the invention said codeine 3-O-demethylase polypeptide comprises an amino acid sequence as set forth in SEQ ID NO: 63, or a polypeptide comprising the amino acid sequence that is at least 90% identical to SEQ ID NO: 63 and includes the amino acid substitution 15K and M360I.

In a preferred embodiment of the invention said codeine 3-O-demethylase polypeptide comprises an amino acid sequence as set forth in SEQ ID NO: 64, or a polypeptide comprising the amino acid sequence that is at least 90% identical to SEQ ID NO: 64 and includes the amino acid substitution 15K and E259G.

In a preferred embodiment of the invention said codeine 3-O-demethylase polypeptide comprises an amino acid sequence as set forth in SEQ ID NO: 65, or a polypeptide comprising the amino acid sequence that is at least 90% identical to SEQ ID NO: 65 and includes the amino acid substitution E259G and M360I.

In a preferred embodiment of the invention said codeine 3-O-demethylase polypeptide comprises an amino acid sequence as set forth in SEQ ID NO: 66, or a polypeptide comprising the amino acid sequence that is at least 90% identical to SEQ ID NO: 66 and includes the amino acid substitution 15K, E259G and M360I.

In a preferred embodiment of the invention said codeine 3-O-demethylase polypeptide comprises an amino acid sequence as set forth in SEQ ID NO: 67, or a polypeptide comprising the amino acid sequence that is at least 90% identical to SEQ ID NO: 67 and includes the amino acid substitution P4A, E259G and M360I.

In a preferred embodiment of the invention said codeine 3-O-demethylase polypeptide comprises an amino acid sequence as set forth in SEQ ID NO: 68, or a polypeptide comprising the amino acid sequence that is at least 90% identical to SEQ ID NO: 68 and includes the amino acid substitution P4A, 15K, E259G and M360I.

In a preferred method of the invention said nucleic acid molecule comprises a nucleotide sequence selected from the group consisting of:
i) a nucleotide sequence as set forth in SEQ ID NO: 16;
ii) a nucleotide sequence wherein said sequence is degenerate as a result of the genetic code to the nucleotide sequence defined in i);
iii) a nucleic acid molecule comprising a nucleotide sequence the complementary strand of which hybridizes under stringent hybridization conditions to the sequence in SEQ ID NO: 16 wherein said nucleic acid molecule encodes a thebaine 6-O-demethylase;
iv) a nucleotide sequence that encodes a polypeptide comprising an amino acid sequence as represented in SEQ ID NO: 32;
v) a nucleotide sequence that encodes a polypeptide comprising an amino acid sequence wherein said amino acid sequence is modified by addition deletion or substitution of at least one amino acid residue as represented in iv) above and which has thebaine 6-O-demethylase activity.

In a preferred method of the invention said nucleic acid molecule comprises a nucleotide sequence selected from the group consisting of:
i) a nucleotide sequence as set forth in SEQ ID NO: 18;
ii) a nucleotide sequence wherein said sequence is degenerate as a result of the genetic code to the nucleotide sequence defined in i);
iii) a nucleic acid molecule comprising a nucleotide sequence the complementary strand of which hybridizes under stringent hybridization conditions to the sequence in SEQ ID NO: 18 wherein said nucleic acid molecule encodes a codeinone reductase;
iv) a nucleotide sequence that encodes a polypeptide comprising an amino acid sequence as represented in SEQ ID NO: 34;
v) a nucleotide sequence that encodes a polypeptide comprising an amino acid sequence wherein said amino acid sequence is modified by addition deletion or substitution of at least one amino acid residue as represented in iv) above and which has codeinone reductase activity.

In a preferred method of the invention of the invention said nucleic acid molecule comprises a nucleotide sequence selected from the group consisting of:
i) a nucleotide sequence as set forth in SEQ ID NO: 17;
ii) a nucleotide sequence wherein said sequence is degenerate as a result of the genetic code to the nucleotide sequence defined in i);
iii) a nucleic acid molecule comprising a nucleotide sequence the complementary strand of which hybridizes under stringent hybridization conditions to the sequence in SEQ ID NO: 17 wherein said nucleic acid molecule encodes a neopinone isomerase;
iv) a nucleotide sequence that encodes a polypeptide comprising an amino acid sequence as represented in SEQ ID NO: 33;
v) a nucleotide sequence that encodes a polypeptide comprising an amino acid sequence wherein said amino acid sequence is modified by addition deletion or substitution of at least one amino acid residue as represented in iv) above and which has neopinone isomerase activity.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, NY, 2001); and Tijssen, Laboratory Techniques in Biochemistry and Molecular Biology-Hybridization with Nucleic Acid Probes Part I, Chapter 2 (Elsevier, New York, 1993). The Tm is the temperature at which 50% of a given strand of a nucleic acid molecule is hybridized to its complementary strand. The following is an exemplary set of hybridization conditions and is not limiting:

Very High Stringency (Allows Sequences that Share at Least 90%, 91%, 92%, 93%, 94%, 95%, 96%, 97%, 98% or 99% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80%, 81%, 82%, 83%, 84%, 85%, 86%, 87%, 88% or 89% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 70%, 71%, 72%, 73%, 74%, 75%, 76%, 77%, 78% or 79% Identity to Hybridize)
Hybridization: 6×SSC at RT to 55° C. for 16-20 hours
Wash at least twice: 2×-3×SSC at RT to 55° C. for 20-30 minutes each.

In a preferred method of the invention said host cell is a eukaryotic cell.

In a preferred method of the invention said eukaryotic cell is a plant cell, for example a Papaver species plant cell e.g., P. somniferum cell.

In a preferred method of the invention said eukaryotic cell is a microbial cell, for example a Saccharomyces cerevisiae cell or Pichia pastoris cell.

In an alternative preferred method of the invention said host cell is a prokaryotic cell.

In a further alternative preferred embodiment of the invention said prokaryotic cell is a bacterial cell, for example an Escherichia coli (E. coli) cell.

If microorganisms are used as organisms in the process according to the invention, they are grown or cultured in the manner with which the skilled worker is familiar, depending on the host organism. As a rule, microorganisms are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen.

The pH of the liquid medium can either be kept constant, regulated during the culturing period, or not. The cultures can be grown batchwise, semi-batchwise, or continuously. Nutrients can be provided at the beginning of the fermentation or fed in semi-continuously or continuously. These products can be isolated from the organisms as described above by processes known to the skilled worker, for example, selective extraction from an aqueous phase into and out of an immiscible organic solvent through the manipulation of product solubilities by pH adjustment with acids or bases. Purification using adsorbents and or formation of crystals by changing the solubility of the target compounds using temperature, polarity of the solution, pH of the solution, formation of salts of the target compounds, thereby allowing isolation of the desired products. To this end, the organisms can advantageously be disrupted beforehand. In this process the pH value is advantageously kept between 4 and 12, preferably between pH 6 and 9, especially preferably between pH 7 and 8.

An overview of known cultivation methods can be found in the textbook by Chmiel (Bioprozeßtechnik 1. Einführung in die Bioverfahrenstechnik [Bioprocess technology 1. Introduction to Bioprocess technology] (Gustav Fischer Verlag, Stuttgart, 1991)) or in the textbook by Storhas (Bioreaktoren und periphere Einrichtungen [Bioreactors and peripheral equipment] (Vieweg Verlag, Brunswick/Wiesbaden, 1994)).

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex mixtures such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogen phosphate or dipotassium hydrogen phosphate or the corresponding sodium-containing salts may be used as sources of phosphorus.

Chelating agents may be added to the medium to keep the metal ions in solution. Particularly suitable chelating agents comprise dihydroxyphenols such as catechol or protocatechuate and organic acids such as citric acid.

The fermentation media used according to the invention for culturing microorganisms usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C. and may be kept constant or may be altered during the experiment. The pH of the medium should be in the range from 5.0 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid, acetic acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air into the culture. The temperature of the culture is normally 20° C. to 45° C. and preferably 25° C. to 40° C. The culture is continued until formation of the desired product is at a maximum. This aim is normally achieved within 10 to 160 hours.

The fermentation broths obtained in this way, those comprising polyunsaturated fatty acids, usually contain a dry mass of from 7.5% to 25% by weight.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation.

However, the fermentation broth can also be thickened or concentrated without separating the cells, using known methods such as, for example, with the aid of a rotary evaporator, thin-film evaporator, falling-film evaporator, by reverse osmosis or by nanofiltration. Finally, this concentrated fermentation broth can be processed to obtain the opiate alkaloids present therein.

In a preferred method of the invention said crude poppy extract is obtained from a Papaver somniferum or Papaver bracteatum plant.

Information on the optimization of extraction methods can be found in the textbook “Natural Product Extraction, Principles and Applications” (Edited by M Rostagno and J Prado, RSC Publishing (2013), ISBN 978 1 84973 606 0).

Preferably, said Papaver species naturally comprises high levels of one or more opiate alkaloid(s).

In an alternative preferred method of the invention said Papaver species is modified wherein said modification is a genomic modification wherein said genomic modification is associated with elevated opiate alkaloid content.

In a preferred method of the invention said Papaver species is P. somniferum and said modification is mutation(s) in one or more codeine 3-O-demethylase.

In a preferred method of the invention said P. somniferum plant is modified wherein the plant is deleted or mutated for one, two or three linked codeine 3-O-demethylase genes encoded by a nucleic acid molecule comprising the nucleotide sequence set forth in SEQ ID NO: 1, or a nucleic acid molecule comprising a nucleotide sequence that is 95-99% identical to the nucleotide sequence set forth in SEQ ID NO: 1.

In a preferred method of the invention said P. somniferum plant is modified wherein the plant is deleted or mutated for one, two or three linked codeine 3-O-demethylase genes encoded by a nucleic acid molecule comprising the nucleotide sequence set forth in SEQ ID NO: 37, or a nucleic acid molecule comprising a nucleotide sequence that is 95-99% identical to the nucleotide sequence set forth in SEQ ID NO: 37.

In a preferred method of the invention said P. somniferum plant is modified by deletion of all or part of a nucleic acid molecule comprising or consisting of the nucleotide sequence as set forth in SEQ ID NO: 35 wherein one, two or three linked codeine 3-O-demethylase genes are deleted or mutated.

In a preferred method of the invention said P. somniferum plant is deleted for each codeine 3-O-demethylase gene wherein codeine 3-O-demethylase activity is undetectable.

The term “gene” is not limited to the open reading frame but can also extend to promoter regions and other regulatory sequences. For example, the activity of an enzyme can be undetectable due to deleted or mutated regulatory regions which results in a lack of gene expression and subsequently enzyme activity.

In a preferred method of the invention said P. somniferum plant comprises a modification wherein said modification is deletion or mutation in one or more thebaine 6-O-demethylases.

In a preferred method of the invention said P. somniferum plant is modified wherein the plant is deleted or mutated for one, two, three, four or five linked thebaine 6-O-demethylase genes encoded by a nucleic acid molecule comprising the nucleotide sequence set forth in SEQ ID NO: 16, or a nucleic acid molecule comprising a nucleotide sequence that is 95-99% identical to the nucleotide sequence set forth in SEQ ID NO: 16.

In a preferred method of the invention said P. somniferum plant is modified by deletion of all or part of a nucleic acid molecule comprising or consisting of the nucleotide sequence as set forth in SEQ ID NO: 36 wherein one, two, three, four or five linked thebaine 6-O-demethylase genes are deleted or mutated.

In a preferred method of the invention said P. somniferum plant is deleted for each thebaine 6-O-demethylase gene wherein 6-O-demethylase activity is undetectable.

In a preferred method of the invention said P. somniferum plant is modified and comprises:
a genomic modification to one, two or three genes encoding codeine 3-O-demethylases,
a genomic modification to one, two, three, four or five genes encoding thebaine 6-O-demethylases,
   wherein the expression of codeine 3-O-demethylases or the activity of codeine 3-O-demethylases is reduced or undetectable and further wherein the expression of said thebaine 6-O-demethylases or activity of thebaine 6-O-demethylases is reduced or undetectable wherein the modified plant has elevated levels of thebaine when compared to a wild type P. somniferum plant and comprising functional genes encoding codeine 3-O-demethylase(s) and functional genes encoding thebaine 6-O-demethylase(s).

According to a further aspect of the invention there is provided a modified codeine 3-O-demethylase polypeptide wherein the activity of said modified codeine 3-O-demethylase polypeptide is altered.

In a preferred embodiment of the invention said modification is to an amino acid sequence as set forth in SEQ ID NO: 19 wherein said polypeptide is modified by addition, deletion or substitution of at least one amino acid residue.

In a preferred embodiment of the invention said modified codeine 3-O-demethylase polypeptide is modified at amino acid position 259.

In an alternative embodiment of the invention said modified codeine 3-O-demethylase polypeptide is modified at amino acid position 260.

In a further alternative preferred embodiment of the invention said modified codeine 3-O-demethylase polypeptide is modified at amino acid position 259 and 260.

In a preferred embodiment of the invention said modified codeine 3-O-demethylase polypeptide is selected from the group consisting of SEQ ID NO: 20, 21, 22, 23, 24, 25, 26, 27, 28, 29, 30 or 31.

In a preferred embodiment of the invention said modification is to an amino acid sequence as set forth in SEQ ID NO: 53 wherein said polypeptide is modified by addition, deletion or substitution of at least one amino acid residue.

In a further alternative preferred embodiment of the invention said modified codeine 3-O-demethylase polypeptide is modified at amino acid position 3.

In a further alternative preferred embodiment of the invention said modified codeine 3-O-demethylase polypeptide is modified at amino acid position 4.

In a further alternative preferred embodiment of the invention said modified codeine 3-O-demethylase polypeptide is modified at amino acid position 5.

In a further alternative preferred embodiment of the invention said modified codeine 3-O-demethylase polypeptide is modified at amino acid position 7.

In a further alternative preferred embodiment of the invention said modified codeine 3-O-demethylase polypeptide is modified at amino acid position 259.

In a further alternative preferred embodiment of the invention said modified codeine 3-O-demethylase polypeptide is modified at amino acid position 357.

In a further alternative preferred embodiment of the invention said modified codeine 3-O-demethylase polypeptide is modified at amino acid position 360.

In a further alternative preferred embodiment of the invention said modified codeine 3-O-demethylase polypeptide is modified at amino acid position 3, 4, 5, 7, 259, 357 and/or 360, or combinations thereof, optionally at positions selected from the group consisting of i) 357 and 360, ii) 5 and 360, iii) 5 and 259, iv) 259 and 360, v) 5, 259 and 360, vi) 4, 259 and 360, vii) 4, 5, 259 and 360, viii) 3, 4, 5 and 7, and ix) 3, 4, 5, 7, 357 and 360.

Preferably, said modified codeine 3-O-demethylase polypeptide is modified at amino acid position 3, 4, 5, 7, 259, 260, 357 and/or 360 or combinations thereof.

In an alternative preferred embodiment of the invention said modified codeine 3-O-demethylase polypeptide is selected from the group consisting of SEQ ID NO: 54, 55, 56, 57, 58, 59, 60, 61, 62, 63, 64, 65, 66, 67 or 68.

In a preferred embodiment of the invention said modified codeine 3-O-demethylase polypeptide has enhanced activity when compared to a wild type codeine 3-O-demethylase polypeptide as represented by the amino acid sequence set forth in SEQ ID NO: 19.

In a preferred embodiment of the invention said enhanced codeine 3-O-demethylase activity is at least 10% higher when compared to a wild-type codeine 3-O-demethylase as represented by the amino acid sequence set forth in SEQ ID NO: 19.

In a preferred embodiment of the invention said enhanced codeine 3-O-demethylase activity is at least 15%, 20%, 25%, 30%, 35%, 40%, 45%, 50%, 60%, 70%, 80%, 90% or 100% higher when compared to a wild-type codeine 3-O-demethylase polypeptide as represented by the amino acid sequence set forth in SEQ ID NO: 19.

In a preferred embodiment of the invention said modified codeine 3-O-demethylase polypeptide is represented by the amino acid sequence set forth in SEQ ID NO: 21.

In a preferred embodiment of the invention said modified codeine 3-O-demethylase polypeptide is represented by the amino acid sequence set forth in SEQ ID NO: 26.

In a preferred embodiment of the invention said modified codeine 3-O-demethylase polypeptide is represented by the amino acid sequence selected from the group consisting of a sequence set forth in SEQ ID NO: 22, 23, 24, 25, 27, 28, 29, 30 and 31.

In a preferred embodiment of the invention said modified codeine 3-O-demethylase polypeptide has enhanced activity when compared to a wild type codeine 3-O-demethylase polypeptide as represented by the amino acid sequence set forth in SEQ ID NO: 53.

In a preferred embodiment of the invention said enhanced codeine 3-O-demethylase activity is at least 10% higher when compared to a wild-type codeine 3-O-demethylase as represented by the amino acid sequence set forth in SEQ ID NO: 53.

In a preferred embodiment of the invention said enhanced codeine 3-O-demethylase activity is at least 15%, 20%, 25%, 30%, 35%, 40%, 45%, 50%, 60%, 70%, 80%, 90% or 100% higher when compared to a wild-type codeine 3-O-demethylase polypeptide as represented by the amino acid sequence set forth in SEQ ID NO: 53.

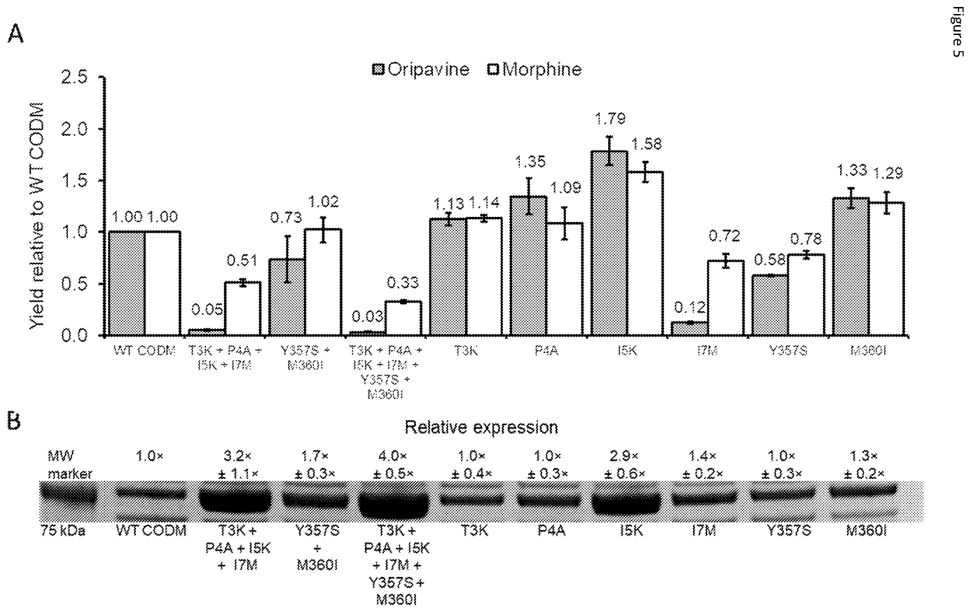

Codeine and thebaine are substrates of the codeine 3-O-demethylase polypeptide. We disclose that the substrate specificity of the codeine 3-O-demethylase polypeptide to codeine and thebaine can change dependent on the specific mutation. This is measurable by a reduced oripavine yield when compared to the yield obtained when using the wild type enzyme and when compared to the morphine yield. See for example FIG. 5. After 4 hours the average thebaine to oripavine yield for the 17M strain was only 6%, vs 47% for the WT CODM expressing strain. While the average codeine to morphine yield was 32% and 47% for the 17M and WT CODM expressing strains, respectively.

In a preferred embodiment of the invention said modified codeine 3-O-demethylase polypeptide has altered substrate specificity when compared to a wild type codeine 3-O-demethylase polypeptide as represented by the amino acid sequence set forth in SEQ ID NO: 53 or 19.

In a preferred embodiment of the invention said altered substrate specificity is represented by an increase or decrease in oripavine yield when compared to the wild type yield.

In a preferred embodiment of the invention said altered substrate specificity is represented by an increase or decrease in morphine yield when compared to the wild type yield.

In a preferred embodiment of the invention SEQ ID NO 54, 55, 56, 60 and 61 show a higher substrate specificity for thebaine when compared to codeine and when compared to the wild type.

In a preferred embodiment of the invention SEQ ID NO 57, 58, 59, 62. 63. 64. 65. 66. 67 and 68 show a higher substrate specificity for codeine when compared to thebaine and when compared to the wild type.

In a preferred embodiment of the invention said modified codeine 3-O-demethylase polypeptide is represented by the amino acid sequence selected from the group consisting of SEQ ID NO 54, 55, 56, 57, 58, 59, 60, 61, 62, 63, 64, 65, 67 and 68.

In a preferred embodiment of the invention said modified codeine 3-O-demethylase polypeptide is represented by the amino acid sequence set forth in SEQ ID NO: 49.

In a preferred embodiment of the invention said modified codeine 3-O-demethylase polypeptide is represented by the amino acid sequence set forth in SEQ ID NO: 55.

In a preferred embodiment of the invention said modified codeine 3-O-demethylase polypeptide is represented by the amino acid sequence set forth in SEQ ID NO: 56.

In a preferred embodiment of the invention said modified codeine 3-O-demethylase polypeptide is represented by the amino acid sequence set forth in SEQ ID NO: 57.

In a preferred embodiment of the invention said modified codeine 3-O-demethylase polypeptide is represented by the amino acid sequence set forth in SEQ ID NO: 58.

In a preferred embodiment of the invention said modified codeine 3-O-demethylase polypeptide is represented by the amino acid sequence set forth in SEQ ID NO: 59.

In a preferred embodiment of the invention said modified codeine 3-O-demethylase polypeptide is represented by the amino acid sequence set forth in SEQ ID NO: 60.

In a preferred embodiment of the invention said modified codeine 3-O-demethylase polypeptide is represented by the amino acid sequence set forth in SEQ ID NO: 61.

In a preferred embodiment of the invention said modified codeine 3-O-demethylase polypeptide is represented by the amino acid sequence set forth in SEQ ID NO: 62.

In a preferred embodiment of the invention said modified codeine 3-O-demethylase polypeptide is represented by the amino acid sequence set forth in SEQ ID NO: 65.

In a preferred embodiment of the invention said modified codeine 3-O-demethylase polypeptide is represented by the amino acid sequence set forth in SEQ ID NO: 64

In a preferred embodiment of the invention said modified codeine 3-O-demethylase polypeptide is represented by the amino acid sequence set forth in SEQ ID NO: 68.

In a preferred embodiment of the invention said modified codeine 3-O-demethylase polypeptide is represented by the amino acid sequence set forth in SEQ ID NO: 67.

In a preferred embodiment of the invention said modified codeine 3-O-demethylase polypeptide is represented by the amino acid sequence set forth in SEQ ID NO: 66.

In a preferred embodiment of the invention said modified codeine 3-O-demethylase polypeptide is represented by the amino acid sequence set forth in SEQ ID NO: 63.

According to a further aspect of the invention there is provided an isolated nucleic acid molecule comprising a nucleotide sequence encoding a modified codeine 3-O-demethylase polypeptide according to the invention.

In a preferred embodiment of the invention said nucleic acid molecule comprises a nucleotide sequence as set forth in SEQ ID NO: 2, 3, 4, 5, 6, 7, 8, 9, 10, 11, 12, 13, 14 or 15 or comprising a nucleotide sequence that is at least 90% identical to SEQ ID NO: 2, 3, 4, 5, 6, 7, 8, 9, 10, 11, 12, 13, 14 or 15 and encodes a polypeptide with modified codeine 3-O-demethylase activity.

In a preferred embodiment of the invention said nucleic acid molecule comprises a nucleotide sequence as set forth in SEQ ID NO: 38, 39, 40, 41, 42, 43, 44, 45, 46, 47, 48, 49, 50, 51 or 52 or comprising a nucleotide sequence that is at least 90% identical to SEQ ID NO: 38, 39, 40, 41, 42, 43, 44, 45, 46, 47, 48, 49, 50, 51 or 52 and encodes a polypeptide with modified codeine 3-O-demethylase activity.

In a preferred embodiment of the invention said nucleic acid molecule comprises a nucleotide sequence as set forth in SEQ ID NO: 1 wherein said sequence is modified wherein said modification is to a codon for amino acid residue 259 and/or 260.

In a preferred embodiment of the invention said nucleic acid molecule comprises a nucleotide sequence as set forth in SEQ ID NO: 37 wherein said sequence is modified wherein said modification is to a codon for amino acid residue 3, 4, 5, 7, 259, 357 and/or 360.

In a preferred embodiment of the invention said nucleic acid molecule comprises a nucleotide sequence encoding modified codeine 3-O-demethylase is set forth in SEQ ID NO: 3 or comprising a nucleotide sequence that is at least 90% identical to SEQ ID NO: 3 and includes a modification to a codon for amino acid residue 259 and optionally also 260.

In a preferred embodiment of the invention said nucleotide sequence encoding modified codeine 3-O-demethylase is set forth in SEQ ID NO: 8 or comprising a nucleotide sequence that is at least 90% identical to SEQ ID NO: 8 and includes a modification to a codon for amino acid residue 259 and optionally also 260.

In a preferred embodiment of the invention said nucleic acid molecule comprises a nucleotide sequence encoding modified codeine 3-O-demethylase is selected from the group consisting of SEQ ID NO: 2, 4, 5, 6, 7, 10, 12, 13, 14 and 15, or comprising a nucleotide sequence that is at least 90% identical to a sequence selected from the group consisting of SEQ ID NO: 2, 4, 5, 6, 7, 10, 12, 13, 14 and 15 and includes a modification to a codon for amino acid residue 259 and optionally also 260.

In a preferred embodiment of the invention said nucleic acid molecule comprises a nucleotide sequence encoding modified codeine 3-O-demethylase is selected from the group consisting of SEQ ID NO: 9 and 11 or comprising a nucleotide sequence that is at least 90% identical to a sequence selected from the group consisting of SEQ ID NO: 9 and 11 and includes a modification to a codon for amino acid residue 260 and optionally also 259.

In a preferred embodiment of the invention said nucleotide sequence encoding modified codeine 3-O-demethylase is selected from the group consisting of SEQ ID NO: 38, 39, 40, 41, 42, 43, 44, 45, 46, 47, 48, 49, 50, 51 and 52, or comprising a nucleotide sequence that is at least 90% identical to a sequence selected from the group consisting of SEQ ID NO: 38, 39, 40, 41, 42, 43, 44, 45, 46, 47, 48, 49, 50, 51 and 52 and includes a modification to a codon for the amino acid residues selected from the group consisting of 3, 4, 5, 7, 259, 357 and 360, and optionally combinations thereof selected from the group consisting of i) 357 and 360, ii) 5 and 360, iii) 5 and 259, iv) 259 and 360, v) 5, 259 and 360, vi) 4, 259 and 360, vii) 4, 5, 259 and 360, viii) 3, 4, 5 and 7, and ix) 3, 4, 5, 7, 357 and 360

In a preferred embodiment of the invention said nucleotide sequence encodes a modified codeine 3-O-demethylase polypeptide comprising an amino acid sequence as set forth in SEQ ID NO: 20, or a polypeptide comprising the amino acid sequence that is at least 90% identical to SEQ ID NO: 20 and includes the amino acid substitution E259K.

In a preferred embodiment of the invention said nucleotide sequence encodes a modified codeine 3-O-demethylase polypeptide comprising an amino acid sequence as set forth in SEQ ID NO: 21, or a polypeptide comprising the amino acid sequence that is at least 90% identical to SEQ ID NO: 21 and includes the amino acid substitution E259D.

In a preferred embodiment of the invention said nucleotide sequence encodes a modified codeine 3-O-demethylase polypeptide comprising an amino acid sequence as set forth in SEQ ID NO: 22 or a polypeptide comprising the amino acid sequence that is at least 90% identical to SEQ ID NO: 22 and includes the amino acid substitution E259H.

In a preferred embodiment of the invention said nucleotide sequence encodes a modified codeine 3-O-demethylase polypeptide comprising an amino acid sequence as set forth in SEQ ID NO: 23, or a polypeptide comprising the amino acid sequence that is at least 90% identical to SEQ ID NO: 23 and includes the amino acid substitution E259Q.

In a preferred embodiment of the invention said nucleotide sequence encodes a modified codeine 3-O-demethylase polypeptide comprising an amino acid sequence as set forth in SEQ ID NO: 24, or a polypeptide comprising the amino acid sequence that is at least 90% identical to SEQ ID NO: 24 and includes the amino acid substitution E259A.

In a preferred embodiment of the invention said nucleotide sequence encodes a modified codeine 3-O-demethylase polypeptide comprising an amino acid sequence as set forth in SEQ ID NO: 25, or a polypeptide comprising the amino acid sequence that is at least 90% identical to SEQ ID NO: 25 and includes the amino acid substitution E259S.

In a preferred embodiment of the invention said nucleotide sequence encodes a modified codeine 3-O-demethylase polypeptide comprising an amino acid sequence as set forth in SEQ ID NO: 26 or a polypeptide comprising the amino acid sequence that is at least 90% identical to SEQ ID NO: 26 and includes the amino acid substitution E259G.

In a preferred embodiment of the invention said nucleotide sequence encodes a modified codeine 3-O-demethylase polypeptide comprising an amino acid sequence as set forth in SEQ ID NO: 27 or a polypeptide comprising the amino acid sequence that is at least 90% identical to SEQ ID NO: 27 and includes the amino acid substitution R260T.

In a preferred embodiment of the invention said nucleotide sequence encodes a modified codeine 3-O-demethylase polypeptide comprising an amino acid sequence as set forth in SEQ ID NO: 28, or a polypeptide comprising the amino acid sequence that is at least 90% identical to SEQ ID NO: 28 and includes the amino acid substitution E259G and R260T.

In a preferred embodiment of the invention said nucleotide sequence encodes a modified codeine 3-O-demethylase polypeptide comprising an amino acid sequence as set forth in SEQ ID NO: 29 or a polypeptide comprising the amino acid sequence that is at least 90% identical to SEQ ID NO: 29 and includes the amino acid substitution R260K.

In a preferred embodiment of the invention said nucleotide sequence encodes a modified codeine 3-O-demethylase polypeptide comprising an amino acid sequence as set forth in SEQ ID NO: 30, or a polypeptide comprising the amino acid sequence that is at least 90% identical to SEQ ID NO: 30 and includes the amino acid substitution E259D and R260K.

In a preferred embodiment of the invention said nucleotide sequence encodes a modified codeine 3-O-demethylase polypeptide comprising an amino acid sequence as set forth in SEQ ID NO: 31 or a polypeptide comprising the amino acid sequence that is at least 90% identical to SEQ ID NO: 31 and includes the amino acid substitution E259G and R260K.

In a preferred embodiment of the invention said nucleotide sequence encodes a modified codeine 3-O-demethylase polypeptide comprising an amino acid sequence as set forth in SEQ ID NO: 54, or a polypeptide comprising the amino acid sequence that is at least 90% identical to SEQ ID NO: 54 and includes the amino acid substitution T3K, P4A, 15K and 17M.

In a preferred embodiment of the invention said nucleotide sequence encodes a modified codeine 3-O-demethylase polypeptide comprising an amino acid sequence as set forth in SEQ ID NO: 55 or a polypeptide comprising the amino acid sequence that is at least 90% identical to SEQ ID NO: 55 and includes the amino acid substitution Y357S and M360I.

In a preferred embodiment of the invention said nucleotide sequence encodes a modified codeine 3-O-demethylase polypeptide comprising an amino acid sequence as set forth in SEQ ID NO: 56, or a polypeptide comprising the amino acid sequence that is at least 90% identical to SEQ ID NO: 56 and includes the amino acid substitution T3K, P4A, 15K, 17M, Y357S and M360I.

In a preferred embodiment of the invention said nucleotide sequence encodes a modified codeine 3-O-demethylase polypeptide comprising an amino acid sequence as set forth in SEQ ID NO: 57 or a polypeptide comprising the amino acid sequence that is at least 90% identical to SEQ ID NO: 57 and includes the amino acid substitution T3K.

In a preferred embodiment of the invention said nucleotide sequence encodes a modified codeine 3-O-demethylase polypeptide comprising an amino acid sequence as set forth in SEQ ID NO: 58, or a polypeptide comprising the amino acid sequence that is at least 90% identical to SEQ ID NO: 58 and includes the amino acid substitution P4A.

In a preferred embodiment of the invention said nucleotide sequence encodes a modified codeine 3-O-demethylase polypeptide comprising an amino acid sequence as set forth in SEQ ID NO: 59 or a polypeptide comprising the amino acid sequence that is at least 90% identical to SEQ ID NO: 59 and includes the amino acid substitution 15K.

In a preferred embodiment of the invention said nucleotide sequence encodes a modified codeine 3-O-demethylase polypeptide comprising an amino acid sequence as set forth in SEQ ID NO: 60, or a polypeptide comprising the amino acid sequence that is at least 90% identical to SEQ ID NO: 60 and includes the amino acid substitution 17M.

In a preferred embodiment of the invention said nucleotide sequence encodes a modified codeine 3-O-demethylase polypeptide comprising an amino acid sequence as set forth in SEQ ID NO: 61 or a polypeptide comprising the amino acid sequence that is at least 90% identical to SEQ ID NO: 61 and includes the amino acid substitution Y357S.

In a preferred embodiment of the invention said nucleotide sequence encodes a modified codeine 3-O-demethylase polypeptide comprising an amino acid sequence as set forth in SEQ ID NO: 62, or a polypeptide comprising the amino acid sequence that is at least 90% identical to SEQ ID NO: 62 and includes the amino acid substitution M360I.

In a preferred embodiment of the invention said nucleotide sequence encodes a modified codeine 3-O-demethylase polypeptide comprising an amino acid sequence as set forth in SEQ ID NO: 63 or a polypeptide comprising the amino acid sequence that is at least 90% identical to SEQ ID NO: 63 and includes the amino acid substitution 15K and M360I.

In a preferred embodiment of the invention said nucleotide sequence encodes a modified codeine 3-O-demethylase polypeptide comprising an amino acid sequence as set forth in SEQ ID NO: 64, or a polypeptide comprising the amino acid sequence that is at least 90% identical to SEQ ID NO: 64 and includes the amino acid substitution 15K and E259G.

In a preferred embodiment of the invention said nucleotide sequence encodes a modified codeine 3-O-demethylase polypeptide comprising an amino acid sequence as set forth in SEQ ID NO: 65 or a polypeptide comprising the amino acid sequence that is at least 90% identical to SEQ ID NO: 65 and includes the amino acid substitution E259G and M360I.

In a preferred embodiment of the invention said nucleotide sequence encodes a modified codeine 3-O-demethylase polypeptide comprising an amino acid sequence as set forth in SEQ ID NO: 66, or a polypeptide comprising the amino acid sequence that is at least 90% identical to SEQ ID NO: 66 and includes the amino acid substitution 15K, E259G and M360I.

In a preferred embodiment of the invention said nucleotide sequence encodes a modified codeine 3-O-demethylase polypeptide comprising an amino acid sequence as set forth in SEQ ID NO: 67 or a polypeptide comprising the amino acid sequence that is at least 90% identical to SEQ ID NO: 67 and includes the amino acid substitution P4A, E259G and M360I.

In a preferred embodiment of the invention said nucleotide sequence encodes a modified codeine 3-O-demethylase polypeptide comprising an amino acid sequence as set forth in SEQ ID NO: 68, or a polypeptide comprising the amino acid sequence that is at least 90% identical to SEQ ID NO: 68 and includes the amino acid substitution P4A, 15K, E259G and M360I.

In a preferred embodiment of the invention said nucleic acid molecule comprises a nucleotide sequence wherein said nucleotide sequence is degenerate because of the genetic code and encodes a modified codeine 3-O-demethylase polypeptide according to the invention.

According to a further aspect of the invention there is provided an expression vector comprising a nucleic acid molecule according to the invention.

In a preferred embodiment of the invention said expression vector comprises a promoter operably linked to said nucleic acid molecule.

In a preferred embodiment of the invention said promoter is a constitutive promoter.

In an alternative preferred embodiment of the invention said promoter is regulatable.

Preferably, said promoter is inducible to provide induced expression of the operably linked nucleic acid.

Preferably said expression vector is adapted for expression in a microbial host cell.

According to an aspect of the invention there is provided a cell transformed with a nucleic acid molecule or vector according to the invention.

In a preferred embodiment of the invention said host cell is a eukaryotic cell.

In a preferred embodiment of the invention said eukaryotic cell is a plant cell, for example a Papaver species plant cell e.g., P. somniferum.

In a preferred embodiment of the invention said eukaryotic cell is a microbial cell, for example a Saccharomyces cerevisiae cell or Pichia pastoris cell.

In an alternative preferred embodiment of the invention said host cell is a prokaryotic cell.

In a further alternative preferred embodiment of the invention said prokaryotic cell is a bacterial cell, for example an Escherichia coli cell.

According to a further aspect of the invention there is provided a cell culture comprising a cell according to the invention.

According to a further aspect of the invention there is provided a fermenter comprising a cell culture according to the invention.

According to a further aspect of the invention there is provided a process for the biotransformation of at first opiate or opioid alkaloid to a second opiate or opioid alkaloid comprising:
forming a preparation comprising a modified codeine 3-O-demethylase polypeptide according to the invention and a crude poppy extract or a purified or semi-purified source of selected opiate or opioid alkaloid; and
incubating the preparation to allow transformation of one or more opiate or opioid alkaloids; and optionally
extracting said one or more transformed opiate or opioid alkaloids from said preparation.

In a preferred method of the invention said modified codeine 3-O-demethylase polypeptide is expressed by a cell according to the invention.

In a preferred method of the invention said first opiate alkaloid is a natural opiate alkaloid.

In an alternative method of the invention said first opiate alkaloid is a semi-synthetic opioid alkaloid.

In a preferred method of the invention said first opiate alkaloid is thebaine and said second opiate alkaloid is oripavine.

In an alternative preferred method of the invention said first opiate alkaloid is codeine and said second opiate alkaloid is morphine.

In a further alternative method of the invention said first opiate alkaloid is codeinone and said second opiate alkaloid is morphinone.

In a further alternative method of the invention said first semi-synthetic opioid alkaloid is oxycodone and said second semi-synthetic opioid alkaloid is oxymorphone.

In a further alternative method of the invention said first semi-synthetic opioid alkaloid is hydrocodone and said second semi-synthetic opioid alkaloid is hydromorphone.

In a further alternative method of the invention said first semi-synthetic opioid alkaloid is dihydrocodeine and said second semi-synthetic opioid alkaloid is dihydromorphine.

In a further alternative method of the invention said first semi-synthetic opioid alkaloid is 14-hydroxycodeine and said second semi-synthetic opioid alkaloid is 14-hydroxymorphine.

In a further alternative method of the invention said first semi-synthetic opioid alkaloid is noroxycodone and said second semi-synthetic opioid alkaloid is noroxymorphone.

In a further alternative method of the invention said first semi-synthetic opioid alkaloid is noroxycodeinone and said second semi-synthetic opioid alkaloid is noroxymorphinone.

In a further alternative method of the invention said first semi-synthetic opioid alkaloid is 14-hydroxy norcodeinone and said second semi-synthetic opioid alkaloid is 14-hydroxy normorphinone.

In a further alternative method of the invention said first semi-synthetic opioid alkaloid is 14-hydroxycodeinone and said second semi-synthetic opioid alkaloid is 14-hydroxymorphinone.

In a further alternative method of the invention said first semi-synthetic opioid alkaloid is buprenorphine intermediate (N-cyclopropylmethyl-7α-(2-hydroxy-3,3-dimethyl-2-butyl)-6,14-endoethano-6,7,8,14-tetrahydronorthebaine) and said second semi-synthetic opioid alkaloid is buprenorphine ((2S)-2-[17-(cyclopropylmethyl)-4,5α-epoxy-3-hydroxy-6-methoxy-6α,14-ethano-14α-morphinan-7α-γl]-3,3-dimethylbutan-2-ol).

In a further alternative method of the invention said first semi-synthetic opioid alkaloid is buprenorphine intermediate (7-acetyl-6,14-endoetheno-6,7,8,14-tetrahydrothebaine) and said second semi-synthetic opioid alkaloid is etorphine intermediate.

Further examples of semi-synthetic opiate alkaloids as disclosed in US2019/0144900 which is incorporated by reference in its entirety.

Throughout the description and claims of this specification, the words “comprise” and “contain” and variations of the words, for example “comprising” and “comprises”, means “includes”, “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures and tables:

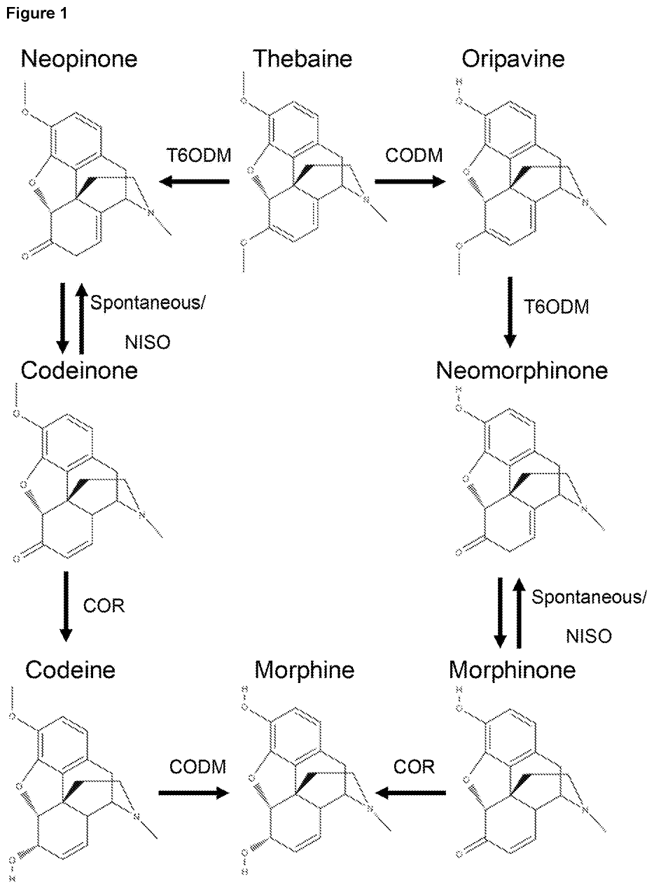

FIG. 1: Pathways for the conversion of thebaine to morphine in Papaver somniferum;

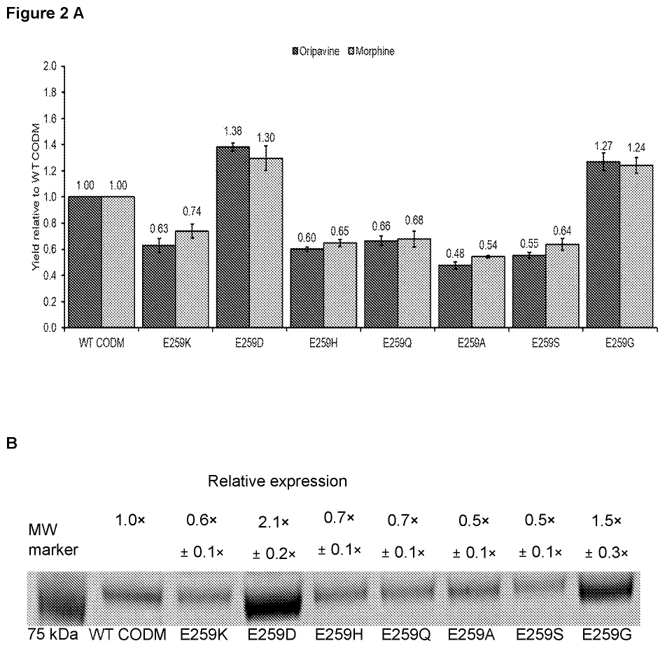

FIG. 2: A) Yield of oripavine and morphine produced by CODM mutants expressed relative to the yield obtained for wild type (WT) CODM. Biotransformations were conducted using strains expressing CODM mutants with varying amino acids at position 259 or WT CODM for 4 hours for oripavine or 30 minutes for morphine. The data are the mean±the standard deviation of three independent replicates. B) Representative SDS-PAGE image of WT CODM and CODM mutant expression. The values give expression relative to WT CODM determined by densiometric analysis, where the data is the mean±the standard deviation of three independent replicates;

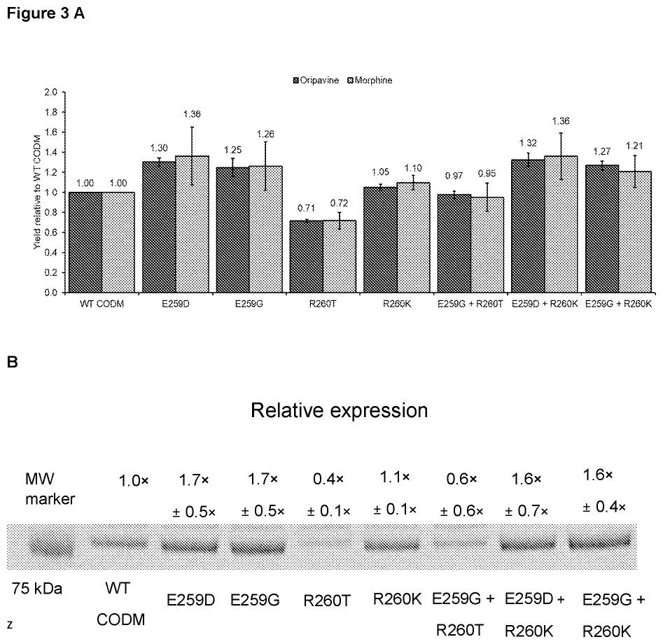

FIG. 3A) Yield of oripavine and morphine produced by CODM mutants expressed relative to the yield obtained for wild type (WT) CODM. Biotransformations were conducted using strains expressing CODM mutants with varying amino acids at position 259, position 260 or both position 259 and 260 or WT CODM for 4 hours for oripavine or 30 minutes for morphine. The data are the mean±the standard deviation of three independent replicates. B) Representative SDS-PAGE image of WT CODM and CODM mutant expression. The values give expression relative to WT CODM determined by densiometric analysis, where the data is the mean±the standard deviation of three independent replicates; and

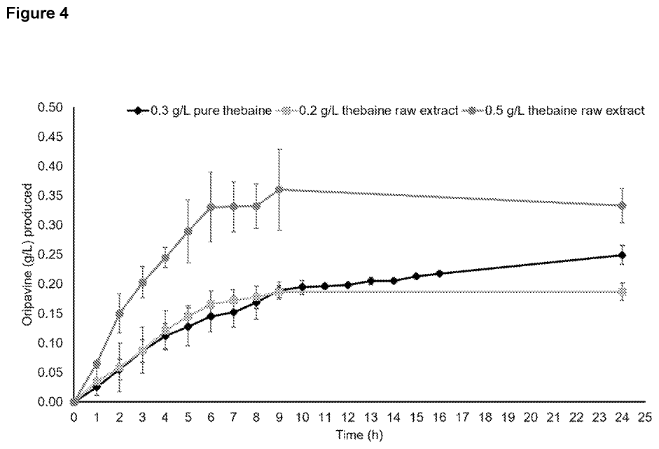

FIG. 4 Production of oripavine as a function of biotransformation progress using pure thebaine or thebaine crude extract as the substrate and cells expressing wild type CODM. Data are the mean±the standard deviation of two independent replicates.

FIG. 5. A) Yield of oripavine and morphine produced by CODM mutants expressed relative to the yield obtained for wild type (WT) CODM. Biotransformations were conducted using strains expressing CODM mutants with a single amino acid residue change (T3K, P4A, 15K, 17M, Y357S or M360I) or a mutation at multiple sites; 1) T3K+P4A+15K+17M, 2) Y357S+M360I, and 3) T3K+P4A+15K+17M+Y357S+M360I or WT CODM. Oripavine yield was assayed after 4 hours and morphine after 30 minutes. The data are the mean±the standard deviation of three independent replicates. B) Representative SDS-PAGE image of WT CODM and CODM mutant protein expression. The values give expression relative to WT CODM determined by densiometric analysis, where the data is the mean±the standard deviation of three independent replicates.

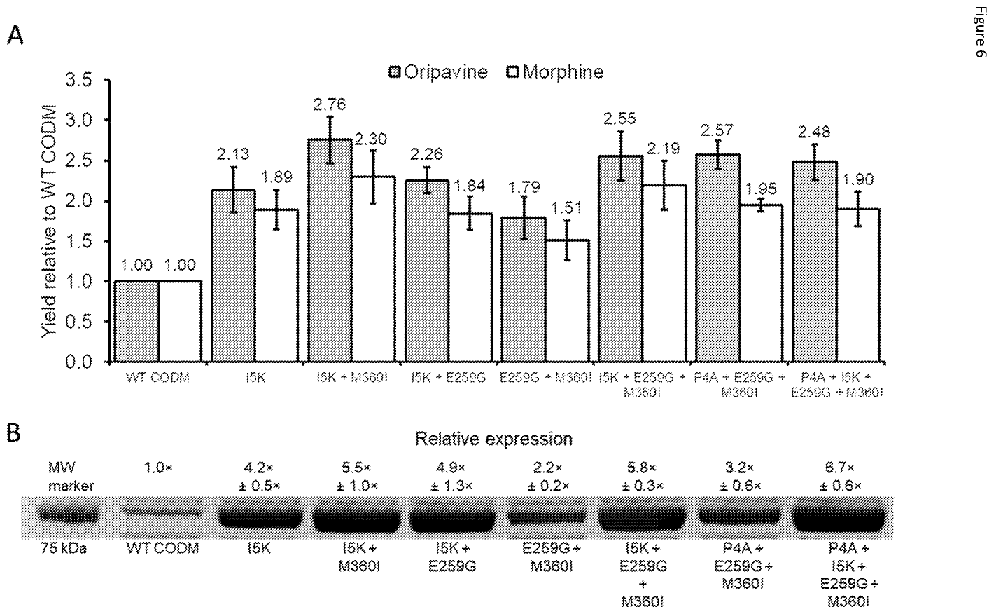

FIG. 6. A) Yield of oripavine and morphine produced by CODM mutants expressed relative to the yield obtained for wild type (WT) CODM. Biotransformations were conducted using strains expressing CODM mutants with combination of the P4A, 15K, E259G and M360I mutations or the 15K single mutation or WT CODM. Oripavine yield was assayed after 4 hours and morphine after 30 minutes. The data are the mean±the standard deviation of three independent replicates. B) Representative SDS-PAGE image of WT CODM and CODM mutant protein expression. The values give expression relative to WT CODM determined by densiometric analysis, where the data is the mean±the standard deviation of three independent replicates.

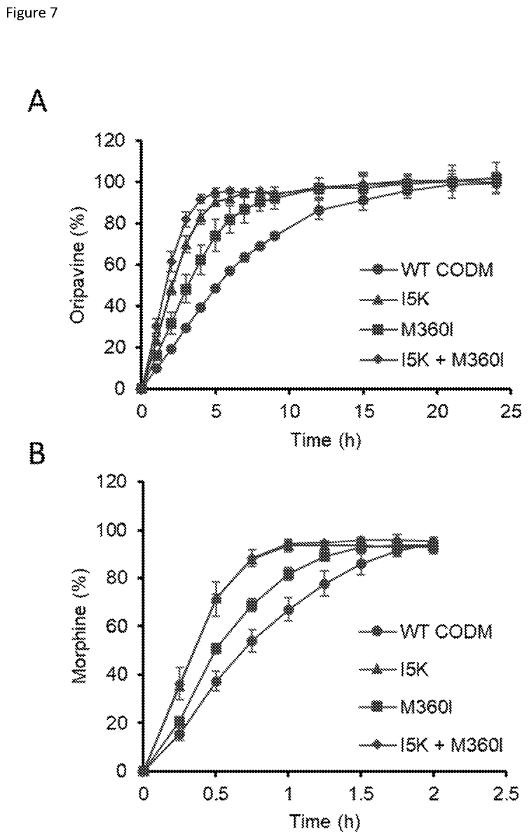

FIG. 7. A) Yield of oripavine produced as a function of time during biotransformation with cells expressing CODM with either the single 15K mutation, the single M360I mutation, the double 15K+M360I mutations or wild type (WT) CODM. Oripavine was assayed every hour for nine hours and then every three hours, until twenty-four hours had passed. B) Yield of morphine produced as a function of time during biotransformation with cells expressing CODM with either the single 15K mutation, the single M360I mutation, the double 15K+M360I mutations or wild type (WT) CODM. Morphine was assayed every fifteen minutes until two hours had passed. The data are the mean±the standard deviation of three independent replicates.

### MATERIALS AND METHODS

### Chemicals and Reagents

Antibiotics, analytical grade glycerol, Isopropyl β-D-1-thiogalactopyranoside (IPTG), sodium chloride, sodium ascorbate, glucose, and iron sulfate heptahydrate, HPLC grade acetonitrile, methanol, trifluoracetic acid (TFA), dichloromethane (DCM) and acetic acid were purchased from Merck (USA). Restriction enzymes, Phusion High Fidelity DNA Polymerase, NEBuilder HiFi DNA Assembly, E. coli 5-alpha competent cells and E. coli BL21 (DE3) competent cells (an E. coli B strain derivative) were purchased from New England BioLabs Inc., USA. Yeast extract and tryptone for medium preparation were purchased from Oxiod, Thermo Scientific, UK. Water for all the experiments was purified to a resistivity of ≥18.2 MΩ·cm. Thebaine, oripavine, codeine and morphine were provided by Sun Pharmaceutical Industries Australia Pty Ltd.

### Plasmids and Stains

Plasmids used in this study are listed in Table 1, primers and synthetic sequences are listed in Table 2 and 3. Synthetic gBlock sequences were codon optimized for expression in E. coli B strains and ordered from Integrated DNA Technologies (IDT, USA). Sufficient vector homology (˜20 bp) for assembly by NEBuilder HiFi DNA Assembly was designed into the gBlocks to speed up construct creation. The T7 polymerase expression vector pET24b-6H-MBP (Merck, USA) was used to control expression of the genes of interest. Each open reading frame (ORF) was N-terminally fused to a six-histidine tag (6H) and a maltose binding protein (MBP). In addition, the ORFs were under the transcriptional control of the T7 promoter and the T7 terminator. E. coli 5-alpha competent cells were used for plasmid cloning and maintenance, while E. coli BL21 (DE3) competent cells were used for biotransformation and protein expression. Assembled constructs were verified by sequencing (Australian Genome Research Facility, Australia) using primers UM13/UM03 (see Table 2).

The CODM (UniProtKB: D4N502) (referred to here as WT CODM for ease of comparison) expression plasmid employing E. coli codon usage, pGWKS100, was created by assembling the 1.1 kb CODM gBlock (UMg4) into the BamHI- and XhoI-linearized pET24b-6H-MBP backbone using NEBuilder.

Several of the expression vectors (pGWKS101, 119-126, see Table 1) were created using the same procedure; the wild type residue was replaced with the appropriate residue by PCR amplification (see Table 2) and then cloned into the linearized pET24b-6H-MBP expression vector using NEBuilder. For example, to construct the expression vector, pGWKS101, of the endogenous CODM E259K variant (NCBI Reference Sequence: XP_026416234.1) the glutamic acid (E) residue present within the CODM WT isoform was replaced with a lysine (K) residue by PCR amplification (UM15/UM05 and UM06/UM18) and the two fragments assembled into linearized pET24b-6H-MBP via NEBuilder.

The remaining expression vectors (pGWSK127-129, 131-146) (see Table 1) were created by assembling the respective 1.1 kb ORF gBlocks (UMg8-26) (see Table 3) into the linearized pET24b-6H-MBP backbone using NEBuilder.

### Cell Culture and Protein Expression

Cells were cultured overnight at 37° C. in Lysogeny Broth medium (0.5% yeast extract, 1% tryptone and 1% NaCl) supplemented with kanamycin (50 μg/mL). 1 mL of the overnight cell culture was inoculated into conical flask that contained 50 mL 2-YT medium (1% yeast extract, 1.6% tryptone and 0.5% NaCl), supplemented with kanamycin (50 μg/mL) and 1 mL glycerol. Protein expression was induced by IPTG addition (0.1 mM) when OD600 (optical density measured at 600 nm) reached 0.4-0.8 and continued for 22 hours at 18° C. Following protein expression cells pellets were collected, by centrifugation at 3,000 g for 15 minutes, and resuspended in 15% glycerol to an OD600 of 100 for biotransformation.

### Whole Cell Biotransformation

Biotransformations were conducted in 20 mL volumes and consisted of the indicated alkaloid (1 mM thebaine, 1 mM codeine or thebaine crude poppy extract), 100 mM phosphate buffer (pH 6.0), 0.5% w/w glucose, 10 UM iron (II) sulfate (FeSO₄) and 10 mM sodium ascorbate and E. coli cells of OD600 of 10. The reaction was conducted at 24° C. with shaking at 220 rpm. Samples were taken regularly at different time intervals. Samples collected were centrifuged, at 16,000 g for 7 minutes, and the supernatant was analyzed by liquid chromatography-mass spectrometer for alkaloid quantification. Three independent replicates were conducted for CODM mutant biotransformations and two for thebaine crude extract biotransformations.

### Protein Expression Analysis

The soluble protein produced by protein expression was determined by SDS-PAGE analysis using the BugBuster™ protocol provided by the manufacturer. Briefly, the soluble protein fraction was liberated from frozen cell pellets, where the OD600 was 1, by resuspension in BugBuster™ protein extraction reagent containing Benzonase (25 units/mL of BugBuster™ reagent) (Merck, Germany). The insoluble cell debris and soluble protein containing supernatant were then separated by centrifugation at 16,000 g for 20 minutes and 4° C. The soluble protein fractions were run on a pre-cast Bolt 8% Bis-Tris Plus Gels (ThermoFisher Scientific, USA) in MOPS running buffer at 120 V for 60 minutes. Protein size was estimated using the Precision Plus Protein™ Kaleidoscope™ Prestained Protein Standard (Bio-Rad Laboratories, USA). Band intensity (less background intensity and normalized to the 75 kDa molecular weight band) was calculated using the Image Lab software (Bio-Rad Laboratories, USA). Soluble protein expression was determined for each of the CODM mutant assays independent replicates.

### Preparation of Crude Poppy Extract

The optimization of extraction methods can be found in the textbook “Natural Product Extraction, Principles and Applications” (Edited by M Rostagno and J Prado, RSC Publishing (2013), ISBN 978 1 84973 606 0). Extracts are prepared as would be by those familiar in the art of natural product extraction where the extraction may contain aqueous and/or hydrocarbon based liquid systems, at temperatures between 0° C. and 100° C., adjusted for pH (between pH 2 and pH 14) using appropriate acidic or alkaline reagents.

### LC-MS Analysis of Alkaloids

Quantification of alkaloids was performed using a Shimadzu LCMS-2020 liquid chromatograph mass spectrometer. Analysis was carried out on an Onyx Monolithic C18 column (100×4.6 mm, Phenomenex Australia Pty Ltd), with a linear gradient of 0-20% buffer B and at a flow rate increased from 1 mL/min to 2.5 mL/min over 10 min at 28° C. [buffer A: 0.1% TFA in water; buffer B: 0.1% TFA in acetonitrile]. The detector wavelength was set at 285 nm with the reference wavelength set at 360 nm. Alkaloid compounds in biotransformation samples were identified by comparing to alkaloid standards, referring to both retention time [morphine at 4.3 min, codeine at 6.9 min, oripavine at 7.5 min and thebaine at 10.1 min] and mass to charge ratio (m/z) [morphine 286, codeine 300, oripavine 298 and thebaine 312]. Shimadzu LabSolutions software was used to integrate the peak area for each compound to quantify the concentration of alkaloid in each sample, by referring to the peak area of alkaloid standard series with concentrations ranging from 25 μg/mL to 500 μg/mL. Samples were injected into the LC-MS with a 5 μL injection volume for analysis.

### Example 1

The suitability of thebaine crude poppy extract as a biotransformation substrate, relative to pure thebaine, was assessed over the course of 24 hours. Two concentrations of thebaine crude extract, equivalent to 0.2 g/L thebaine and 0.5 g/L thebaine, were assayed. Crude extract was added to the reaction mixture in place of pure thebaine and all other components were kept constant. As can be seen in FIG. 4 thebaine crude extract can replace pure thebaine with minimal variation to reaction progression. Moreover, several concentrations of thebaine crude extract can be employed as a biotransformation substrate, producing higher concentrations of oripavine.

### Example 2

Seven strains were generated containing mutations at the 259^{th} amino acid residue (E259K, -D, -H, -Q, -A, -S and -G) of CODM and two strains were generated that contained mutations at the 260^{th} residue (R260T and -K) of CODM. Three double mutant strains were created containing mutations to both the 259^{th} and 260^{th} residues of CODM (E259G+R260T, E259D+R260K and E259G+R260K). The performance of the CODM mutant strains was assessed via biotransformation with morphine yield assayed after 30 minutes and oripavine yield assayed after 4 hours, as shown in FIG. 2-3. Soluble protein expression under these conditions was assayed via SDS-PAGE analysis, as shown in FIG. 2-3.

Of the seven 259 residue mutant strains investigated, only the E259D and E259G mutants demonstrated an improvement in product yield, with morphine yield improved by ˜30% and ˜24% respectively and oripavine yield by ˜38% and ˜27% respectively relative to the wild type (WT) CODM. Moreover, these mutations appeared to improve the amount of soluble protein expressed with ˜2.1× more soluble E259D and ˜1.5× more soluble E259G produced compared to WT CODM. In comparison, biotransformation with E259K, -H, -Q, -A and -S CODM mutants lead to a reduction in product yield by at least 20% relative to WT CODM, coincident with a reduction in the expression of soluble protein expression, as shown in FIG. 2-3.

The R260T expressing strain generated ˜28% less morphine and ˜29% less oripavine than WT CODM and expressed ˜63% less soluble protein, see FIG. 3. In comparison, the R260K expressing strain produced similar levels of both products to WT CODM, with a similar amount of soluble protein produced to the WT CODM strain, see FIG. 3.

Incorporation of the E259G mutation rescued the performance of the R260T mutation in the E259G+R260T double mutant strain, generating a similar level of conversion to the WT CODM strain (FIG. 3). In comparison, the performance of the E259D+R260K, and E259G+R260K double mutant strains was improved compared to the WT CODM stain and had a performance and level of protein expression comparable to the E259D and E259G single mutant strains (FIG. 3).

### Example 3

Six strains were generated containing a single amino acid mutation at either the 3^{rd}, 4^{th}, 5^{th}, 7^{th}, 357^{th} or 360^{th} residue of WT CODM: T3K, P4A, 15K, 17M, Y357S or M360I. Three additional strains were generated with the following combinations of mutations: 1) T3K+P4A+15K+17M, 2) Y357S+M360I, and 3) T3K+P4A+15K+17M+Y357S+M360I. The performance of the CODM mutant strains was assessed via biotransformation with morphine yield assayed after 30 minutes and oripavine yield assayed after 4 hours, as shown in FIG. 5A. Soluble protein expression under these conditions was assayed via SDS-PAGE analysis, as shown in FIG. 5B.

Of the six single mutant strains investigated, both the 15K strain and the M360I strain demonstrated an improvement in product yield, with morphine yield improved by ˜58% and ˜ 29% respectively and oripavine yield by ˜79% and ˜33% respectively, compared to the WT CODM strain (FIG. 5A). The 15K mutation appeared to improve the amount of soluble protein expressed, with ˜2.9× greater than WT CODM but only small changes in protein expression were observed in the M360I strain compared to WT CODM (FIG. 5B).

The four other single mutant strains (T3K, P4A, 17M or Y357S) led to either a minor improvement in product yield (e.g., T3K or P4A) or a reduction in product yield by at least ˜22% (e.g. 17M or Y357S) relative to the WT CODM strain. This coincided with little change (e.g. T3K, P4A, Y357S or 17M) in the level of protein expression (FIG. 5B).

All three strains containing the combined mutants had a lower biotransformation yield for at least one of the two desired products relative to the WT CODM strain. This coincided with an increase in the level of protein expression for all three strains, which was most notable for the T3K+P4A+15K+17K strain (˜3.2×) and the T3K+P4A+15K+17M+Y357S+M360I strain (˜4×).

### Example 4

Six additional strains were generated containing various combinations of the P4A, 15K, E259G and M360 mutations to assess the impact of these beneficial mutations on the yield and level of soluble protein expression for the modified CODM enzymes. In this case, morphine production was measured after 15 minutes (compared to 30 minutes in Example 3) and oripavine after 2 hours (compared to 4 hours in Example 3) as shown in FIG. 6A. This was due to an accelerated reaction rate for these mutants.

All six strains demonstrated an improvement in yield for both morphine and oripavine production by biotransformation and the level of soluble protein expression detected relative to WT CODM (FIGS. 6A and B). The strain containing both the 15K+M360I mutations produced the highest yield of all the mutant strains, with a 2.3× increase in morphine yield and 2.76× increase in oripavine yield relative to the WT CODM strain (FIG. 6A), with ˜5.5× more soluble protein compared to WT CODM (FIG. 6B).

### Example 5

The performance of the strains containing the single mutations 15K or M360I or the double mutations 15K+M360I was compared to the WT CODM over the time course of the entire thebaine or codeine demethylation reaction. Oripavine was assayed every hour for the first nine hours and then every three hours until twenty-four hours had passed. The quicker codeine demethylation reaction was assessed by measuring morphine every fifteen minutes over two hours.

The strain containing the 15K+M360I double mutation performed the fastest biotransformation, with thebaine as a substrate, achieving a yield of thebaine to oripavine of ˜95% after ˜five hours (FIG. 7A). The strains with single mutations also performed faster than the strain with WT CODM. The bioconversion reaction neared complete conversion for all three mutant strains at least twelve hours before that of the WT CODM strain.

Two strains displayed a similar fast biotransformation rate with codeine as the substrate; these contained the single mutation 15K or the double mutation 15K+M360I, which achieved a yield of morphine of ˜ 94% after one hour, which was faster than the WT CODM control (FIG. 7B). The single mutant M360I was faster than the WT CODM control but not as fast as the other two mutant strains. All three mutants achieved near complete conversion by two hours.

TABLE 1
Plasmids used in this study
All strains were made in pET24b-6H-MBP

| Plasmid | Genotype | Source |
|---|---|---|
| pET24b-6H- | T7 expression vector | Merck |
| MBP | | |
| pGWKS100 | CODM WT cassette in pET24b-6H-MBP | This study |
| pGWKS101 | E259K cassette in pET24b-6H-MBP | This study |
| pGWKS119 | CODM E259D in pET24b-6H-MBP | This study |
| pGWKS120 | CODM E259H in pET24b-6H-MBP | This study |
| pGWKS121 | CODM E259Q in pET24b-6H-MBP | This study |
| pGWKS122 | CODM E259A in pET24b-6H-MBP | This study |
| pGWKS123 | CODM E259S in pET24b-6H-MBP | This study |
| pGWKS124 | CODM E259G in pET24b-6H-MBP | This study |
| pGWKS125 | CODM R260T in pET24b-6H-MBP | This study |
| pGWSK126 | CODM E259G + R260T in pET24b-6H-MBP | This study |
| pGWKS131 | CODM R260K in pET24b-6H-MBP | This study |
| pGWKS132 | CODM E259D + R260K in pET24b-6H-MBP | This study |
| PGWKS133 | CODM E259G + R260K in pET24b-6H-MBP | This study |
| pGWKS134 | CODM WT* cassette in pET24b-6H-MBP | This study |
| pGWKS127 | CODM T3K + P4A + I5K + I7M in pET24b-6H-MBP | This study |
| pGWKS128 | CODM Y357S + M360I in pET24b-6H-MBP | This study |
| pGWKS129 | CODM T3K + P4A + I5K + I7M + Y357S + M360I | This study |
| | in pET24b-6H-MBP | |
| pGWKS135 | CODM T3K in pET24b-6H-MBP | This study |
| pGWKS136 | CODM P4A in pET24b-6H-MBP | This study |
| pGWKS137 | CODM I5K in pET24b-6H-MBP | This study |
| pGWKS138 | CODM I7M in pET24b-6H-MBP | This study |
| pGWKS139 | CODM Y357S in pET24b-6H-MBP | This study |
| pGWSK140 | CODM M360I in pET24b-6H-MBP | This study |
| pGWKS141 | CODM I5K + M360I in pET24b-6H-MBP | This study |
| pGWKS142 | CODM I5K + E259G in pET24b-6H-MBP | This study |
| pGWKS143 | CODM E259G + M360I in pET24b-6H-MBP | This study |
| pGWKS144 | CODM I5K + E259G + M360I in pET24b-6H-MBP | This study |
| pGWKS145 | CODM P4A + E259G + M360I in pET24b-6H-MBP | This study |
| pGWKS146 | CODM P4A + I5K + E259G + M360I in pET24b-6H- | This study |
| | MBP | |

pGWKS134 encodes an identical protein to the WT CODM enzyme within the pGWKS100 plasmid, differing only in the codons used at the 2^{nd} (GAG→GAA), 6^{th} (TTG→CTG) and 118^{th} (CGC→CGT) codon. pGWKS127-129 and pGWKS135-145 contain the equivalent codon usage to pGWKS134 and the pGWKS134 strain was used as the control WT CODM in these assays. This control was used for experiments described in Examples 4-6 but not Examples 1-3, which used pGWKS100 strain as the control WT CODM.

TABLE 2
Primers used in this study

| | | | SEQ |
|---|---|---|---|
| Primer | | | ID |
| ID | Name | Sequence | NO: |
| UM13 | Sequencing | GAAATCATGCCGAACATCCC | 69 |
| | upper | | |
| UM03 | Sequencing | CCGGATATAGTTCCTCCTTTCAGC | 70 |
| | lower | | |
| UM05 | CODM E259K | TCCACCGTTTTTCCTTCCGAAT | 71 |
| | fragment 1 | | |
| | lower | | |
| UM06 | CODM E259K | GATTCGGAAGGAAAAACGGTGGAT | 72 |
| | fragment 2 | C | |
| | upper | | |
| UM15 | CODM E2594 | CGGTCGTCAGACTGTCGATGA | 73 |
| | fragment 1 | | |
| | upper | | |
| UM18 | CODM E2594 | TTAGCAGCCGGATCTCAGTG | 74 |
| | fragment 2 | | |
| | lower | | |
| UM19 | CODM E259D | TCCACCGGTCTTCCTTCCGAAT | 75 |
| | fragment 1 | | |
| | lower | | |
| UM20 | CODM E259D | GATTCGGAAGGAAGACCGGTGGAT | 76 |
| | fragment 2 | C | |
| | upper | | |
| UM21 | CODM E259H | TCCACCGATGTTCCTTCCGAAT | 77 |
| | fragment 1 | | |
| | lower | | |
| UM22 | CODM E259H | GATTCGGAAGGAACATCGGTGGAT | 78 |
| | fragment 2 | C | |
| | upper | | |
| UM23 | CODM E259Q | TCCACCGTTGTTCCTTCCGAAT | 79 |
| | fragment 1 | | |
| | lower | | |
| UM24 | CODM E259Q | GATTCGGAAGGAACAACGGTGGAT | 80 |
| | fragment 2 | C | |
| | upper | | |
| UM25 | CODM E259A | TCCACCGTGCTTCCTTCCGAAT | 81 |
| | fragment 1 | | |
| | lower | | |
| UM26 | CODM E259A | GATTCGGAAGGAAGCACGGTGGAT | 82 |
| | fragment 2 | C | |
| | upper | | |
| UM27 | CODM E259S | TCCACCGACTTTCCTTCCGAAT | 83 |
| | fragment 1 | | |
| | lower | | |
| UM28 | CODM E259S | GATTCGGAAGGAAAGTCGGTGGAT | 84 |
| | fragment 2 | C | |
| | upper | | |
| UM32 | CODM E259G | TCCACCGACCTTCCTTCCGAAT | 85 |
| | fragment 1 | | |
| | lower | | |
| UM33 | CODM E259G | GATTCGGAAGGAAGGTCGGTGGAT | 86 |
| | fragment 2 | C | |
| | upper | | |
| UM34 | CODM R260T | TCCACGTCTCTTCCTTCCGAAT | 87 |
| | fragment 1 | | |
| | lower | | |
| UM35 | CODM R260T | GATTCGGAAGGAAGAGACGTGGAT | 88 |
| | fragment 2 | C | |
| | upper | | |
| UM36 | CODM E259G + | TCCACGTACCTTCCTTCCGAAT | 89 |
| | R260T | | |
| | fragment 1 | | |
| | lower | | |
| UM37 | CODM E259G + | GATTCGGAAGGAAGGTACGTGGAT | 90 |
| | R260T | C | |
| | fragment 2 | | |
| | upper | | |

The “Δ” primers are generic and thus were used as the upper primer (UM15) and lower primer (UM18) for all fragment 1 and 2 amplification, respectively.

The R260K, E259D+R260K and E259G+R260K expression plasmids were created using synthetic sequences rather than via PCR mutagenesis. See materials & methods section.

TABLE 3
Open Reading Frames and Synthetic Sequences

Synthetic sequences used for cloning are denoted by UMgX,
were ordered from IDT and codon optimized for expression in
E. coli B strains. Homology regions
CCTGAAAGACGCGCAGACTGGATCC (SEQ ID NO: 91) and
CTCGAGCACCACCACCACCACCACTGAGATCCGGCTGCTA (SEQ ID NO: 92)
and other similar sequences were included upstream and
downstream, respectively, of each synthetic sequences to
facilitate NEBuilder assembly and are optional in relation to
nucleic acid molecules encoding proteins according to the invention.

| Name | Sequence |
|---|---|
| CODM WT | CCTGAAAGACGCGCAGACTGGATCCATGGAGACCCCAATCTTGATC |
| (UMg4) | AAACTTGGCAACGGGCTCTCGATCCCTAGCGTCCAAGAGCTCGCCA |
| SEQ ID | AGTTGACCCTGGCCGAGATCCCAAGTCGGTATACATGCACAGGTGA |
| NO: 1 | GAGCCCACTTAACAACATCGGTGCGTCAGTAACAGACGATGAAACG |
| | GTGCCGGTCATTGATTTGCAAAACTTATTAAGTCCAGAGCCAGTAGT |
| | GGGGAAATTAGAGTTGGACAAGTTACACTCCGCTTGCAAAGAGTGG |
| | GGCTTCTTTCAGCTTGTCAACCATGGCGTTGATGCCTTGTTAATGGA |
| | CAACATTAAGAGCGAAATCAAGGGCTTTTTTAACCTGCCGATGAATG |
| | AGAAGACCAAATACGGTCAGCAGGACGGCGACTTTGAAGGGTTCG |
| | GTCAGCCTTATATTGAATCTGAGGATCAGCGCTTGGATTGGACTGA |
| | GGTGTTCTCAATGCTCTCGCTGCCACTTCACCTGCGCAAGCCGCAC |
| | CTTTTTCCAGAACTTCCACTTCCGTTTCGCGAGACCCTGGAGTCGTA |
| | CTTGAGCAAGATGAAAAAACTGTCAACGGTGGTGTTTGAGATGTTA |
| | GAAAAGAGTTTGCAACTTGTTGAGATTAAAGGTATGACTGACTTGTT |
| | CGAAGACGGGCTCCAAACGATGCGCATGAATTACTATCCTCCATGT |
| | CCACGGCCTGAGTTGGTATTGGGTCTTACAAGTCATAGTGACTTTTC |
| | TGGGTTGACCATTTTACTCCAGTTAAACGAAGTGGAGGGGTTGCAG |
| | ATTCGGAAGGAAGAGCGGTGGATCAGCATCAAACCGCTTCCAGAC |
| | GCCTTTATTGTCAACGTAGGTGATATTCTCGAAATTATGACCAACGG |
| | GATCTATCGTAGTGTTGAGCACCGTGCAGTCGTGAATAGTACCAAG |
| | GAGCGGCTTTCCATCGCCACATTCCATGACTCTAAATTGGAATCCG |
| | AAATCGGTCCTATCTCTTCGTTGGTTACTCCTGAGACCCCTGCATTA |
| | TTCAAGCGCGGGCGCTACGAAGACATTCTCAAGGAAAACCTTTCGC |
| | GCAAACTTGATGGCAAGTCTTTCCTGGATTACATGCGCATGTGACT |
| | CGAGCACCACCACCACCACCACTGAGATCCGGCTGCTA |
| CODM | ATGGAGACCCCAATCTTGATCAAACTTGGCAACGGGCTCTCGATCC |
| E259K | CTAGCGTCCAAGAGCTCGCCAAGTTGACCCTGGCCGAGATCCCAA |
| SEQ ID | GTCGGTATACATGCACAGGTGAGAGCCCACTTAACAACATCGGTGC |
| NO: 2 | GTCAGTAACAGACGATGAAACGGTGCCGGTCATTGATTTGCAAAAC |
| | TTATTAAGTCCAGAGCCAGTAGTGGGGAAATTAGAGTTGGACAAGT |
| | TACACTCCGCTTGCAAAGAGTGGGGCTTCTTTCAGCTTGTCAACCA |
| | TGGCGTTGATGCCTTGTTAATGGACAACATTAAGAGCGAAATCAAG |
| | GGCTTTTTTAACCTGCCGATGAATGAGAAGACCAAATACGGTCAGC |
| | AGGACGGCGACTTTGAAGGGTTCGGTCAGCCTTATATTGAATCTGA |
| | GGATCAGCGCTTGGATTGGACTGAGGTGTTCTCAATGCTCTCGCTG |
| | CCACTTCACCTGCGCAAGCCGCACCTTTTTCCAGAACTTCCACTTC |
| | CGTTTCGCGAGACCCTGGAGTCGTACTTGAGCAAGATGAAAAAACT |
| | GTCAACGGTGGTGTTTGAGATGTTAGAAAAGAGTTTGCAACTTGTTG |
| | AGATTAAAGGTATGACTGACTTGTTCGAAGACGGGCTCCAAACGAT |
| | GCGCATGAATTACTATCCTCCATGTCCACGGCCTGAGTTGGTATTG |
| | GGTCTTACAAGTCATAGTGACTTTTCTGGGTTGACCATTTTACTCCA |
| | GTTAAACGAAGTGGAGGGGTTGCAGATTCGGAAGGAAAAACGGTG |
| | GATCAGCATCAAACCGCTTCCAGACGCCTTTATTGTCAACGTAGGT |
| | GATATTCTCGAAATTATGACCAACGGGATCTATCGTAGTGTTGAGCA |
| | CCGTGCAGTCGTGAATAGTACCAAGGAGCGGCTTTCCATCGCCACA |
| | GGTTACTCCTGAGACCCCTGCATTATTCAAGCGCGGGCGCTACGAA |
| | GACATTCTCAAGGAAAACCTTTCGCGCAAACTTGATGGCAAGTCTTT |
| | CCTGGATTACATGCGCATGTGA |
| CODM | ATGGAGACCCCAATCTTGATCAAACTTGGCAACGGGCTCTCGATCC |
| E259D | CTAGCGTCCAAGAGCTCGCCAAGTTGACCCTGGCCGAGATCCCAA |
| SEQ ID | GTCGGTATACATGCACAGGTGAGAGCCCACTTAACAACATCGGTGC |
| NO: 3 | GTCAGTAACAGACGATGAAACGGTGCCGGTCATTGATTTGCAAAAC |
| | TTATTAAGTCCAGAGCCAGTAGTGGGGAAATTAGAGTTGGACAAGT |
| | TACACTCCGCTTGCAAAGAGTGGGGCTTCTTTCAGCTTGTCAACCA |
| | TGGCGTTGATGCCTTGTTAATGGACAACATTAAGAGCGAAATCAAG |
| | GGCTTTTTTAACCTGCCGATGAATGAGAAGACCAAATACGGTCAGC |
| | AGGACGGCGACTTTGAAGGGTTCGGTCAGCCTTATATTGAATCTGA |
| | GGATCAGCGCTTGGATTGGACTGAGGTGTTCTCAATGCTCTCGCTG |
| | CCACTTCACCTGCGCAAGCCGCACCTTTTTCCAGAACTTCCACTTC |
| | CGTTTCGCGAGACCCTGGAGTCGTACTTGAGCAAGATGAAAAAACT |
| | GTCAACGGTGGTGTTTGAGATGTTAGAAAAGAGTTTGCAACTTGTTG |
| | AGATTAAAGGTATGACTGACTTGTTCGAAGACGGGCTCCAAACGAT |
| | GCGCATGAATTACTATCCTCCATGTCCACGGCCTGAGTTGGTATTG |
| | GGTCTTACAAGTCATAGTGACTTTTCTGGGTTGACCATTTTACTCCA |
| | GTTAAACGAAGTGGAGGGGTTGCAGATTCGGAAGGAAGACCGGTG |
| | GATCAGCATCAAACCGCTTCCAGACGCCTTTATTGTCAACGTAGGT |
| | GATATTCTCGAAATTATGACCAACGGGATCTATCGTAGTGTTGAGCA |
| | CCGTGCAGTCGTGAATAGTACCAAGGAGCGGCTTTCCATCGCCACA |
| | TTCCATGACTCTAAATTGGAATCCGAAATCGGTCCTATCTCTTCGTT |
| | GGTTACTCCTGAGACCCCTGCATTATTCAAGCGCGGGCGCTACGAA |
| | GACATTCTCAAGGAAAACCTTTCGCGCAAACTTGATGGCAAGTCTTT |
| | CCTGGATTACATGCGCATGTGA |
| CODM | ATGGAGACCCCAATCTTGATCAAACTTGGCAACGGGCTCTCGATCC |
| E259H | CTAGCGTCCAAGAGCTCGCCAAGTTGACCCTGGCCGAGATCCCAA |
| SEQ ID | GTCGGTATACATGCACAGGTGAGAGCCCACTTAACAACATCGGTGC |
| NO: 4 | GTCAGTAACAGACGATGAAACGGTGCCGGTCATTGATTTGCAAAAC |
| | TTATTAAGTCCAGAGCCAGTAGTGGGGAAATTAGAGTTGGACAAGT |
| | TACACTCCGCTTGCAAAGAGTGGGGCTTCTTTCAGCTTGTCAACCA |
| | TGGCGTTGATGCCTTGTTAATGGACAACATTAAGAGCGAAATCAAG |
| | GGCTTTTTTAACCTGCCGATGAATGAGAAGACCAAATACGGTCAGC |
| | AGGACGGCGACTTTGAAGGGTTCGGTCAGCCTTATATTGAATCTGA |
| | GGATCAGCGCTTGGATTGGACTGAGGTGTTCTCAATGCTCTCGCTG |
| | CCACTTCACCTGCGCAAGCCGCACCTTTTTCCAGAACTTCCACTTC |
| | CGTTTCGCGAGACCCTGGAGTCGTACTTGAGCAAGATGAAAAAACT |
| | GTCAACGGTGGTGTTTGAGATGTTAGAAAAGAGTTTGCAACTTGTTG |
| | AGATTAAAGGTATGACTGACTTGTTCGAAGACGGGCTCCAAACGAT |
| | GCGCATGAATTACTATCCTCCATGTCCACGGCCTGAGTTGGTATTG |
| | GGTCTTACAAGTCATAGTGACTTTTCTGGGTTGACCATTTTACTCCA |
| | GTTAAACGAAGTGGAGGGGTTGCAGATTCGGAAGGAACATCGGTG |
| | GATCAGCATCAAACCGCTTCCAGACGCCTTTATTGTCAACGTAGGT |
| | GATATTCTCGAAATTATGACCAACGGGATCTATCGTAGTGTTGAGCA |
| | CCGTGCAGTCGTGAATAGTACCAAGGAGCGGCTTTCCATCGCCACA |
| | TTCCATGACTCTAAATTGGAATCCGAAATCGGTCCTATCTCTTCGTT |
| | GGTTACTCCTGAGACCCCTGCATTATTCAAGCGCGGGCGCTACGAA |
| | GACATTCTCAAGGAAAACCTTTCGCGCAAACTTGATGGCAAGTCTTT |
| | CCTGGATTACATGCGCATGTGA |
| CODM | ATGGAGACCCCAATCTTGATCAAACTTGGCAACGGGCTCTCGATCCCTAGC |
| E259Q | GTCCAAGAGCTCGCCAAGTTGACCCTGGCCGAGATCCCAAGTCGGTATAC |
| SEQ ID | ATGCACAGGTGAGAGCCCACTTAACAACATCGGTGCGTCAGTAACAGACG |
| NO: 5 | ATGAAACGGTGCCGGTCATTGATTTGCAAAACTTATTAAGTCCAGAGCCAG |
| | TAGTGGGGAAATTAGAGTTGGACAAGTTACACTCCGCTTGCAAAGAGTGG |
| | GGCTTCTTTCAGCTTGTCAACCATGGCGTTGATGCCTTGTTAATGGACAAC |
| | ATTAAGAGCGAAATCAAGGGCTTTTTTAACCTGCCGATGAATGAGAAGACC |
| | AAATACGGTCAGCAGGACGGCGACTTTGAAGGGTTCGGTCAGCCTTATATT |
| | GAATCTGAGGATCAGCGCTTGGATTGGACTGAGGTGTTCTCAATGCTCTCG |
| | CTGCCACTTCACCTGCGCAAGCCGCACCTTTTTCCAGAACTTCCACTTCCG |
| | TTTCGCGAGACCCTGGAGTCGTACTTGAGCAAGATGAAAAAACTGTCAACG |
| | GTGGTGTTTGAGATGTTAGAAAAGAGTTTGCAACTTGTTGAGATTAAAGGTA |
| | TGACTGACTTGTTCGAAGACGGGCTCCAAACGATGCGCATGAATTACTATC |
| | CTCCATGTCCACGGCCTGAGTTGGTATTGGGTCTTACAAGTCATAGTGACT |
| | TTTCTGGGTTGACCATTTTACTCCAGTTAAACGAAGTGGAGGGGTTGCAGA |
| | TTCGGAAGGAACAACGGTGGATCAGCATCAAACCGCTTCCAGACGCCTTTA |
| | TTGTCAACGTAGGTGATATTCTCGAAATTATGACCAACGGGATCTATCGTA |
| | GTGTTGAGCACCGTGCAGTCGTGAATAGTACCAAGGAGCGGCTTTCCATC |
| | GCCACATTCCATGACTCTAAATTGGAATCCGAAATCGGTCCTATCTCTTCGT |
| | TGGTTACTCCTGAGACCCCTGCATTATTCAAGCGCGGGCGCTACGAAGAC |
| | ATTCTCAAGGAAAACCTTTCGCGCAAACTTGATGGCAAGTCTTTCCTGGATT |
| | ACATGCGCATGTGA |
| CODM | ATGGAGACCCCAATCTTGATCAAACTTGGCAACGGGCTCTCGATCC |
| E259A | CTAGCGTCCAAGAGCTCGCCAAGTTGACCCTGGCCGAGATCCCAA |
| SEQ ID | GTCGGTATACATGCACAGGTGAGAGCCCACTTAACAACATCGGTGC |
| NO: 6 | GTCAGTAACAGACGATGAAACGGTGCCGGTCATTGATTTGCAAAAC |
| | TTATTAAGTCCAGAGCCAGTAGTGGGGAAATTAGAGTTGGACAAGT |
| | TACACTCCGCTTGCAAAGAGTGGGGCTTCTTTCAGCTTGTCAACCA |
| | TGGCGTTGATGCCTTGTTAATGGACAACATTAAGAGCGAAATCAAG |
| | GGCTTTTTTAACCTGCCGATGAATGAGAAGACCAAATACGGTCAGC |
| | AGGACGGCGACTTTGAAGGGTTCGGTCAGCCTTATATTGAATCTGA |
| | GGATCAGCGCTTGGATTGGACTGAGGTGTTCTCAATGCTCTCGCTG |
| | CCACTTCACCTGCGCAAGCCGCACCTTTTTCCAGAACTTCCACTTC |
| | CGTTTCGCGAGACCCTGGAGTCGTACTTGAGCAAGATGAAAAAACT |
| | GTCAACGGTGGTGTTTGAGATGTTAGAAAAGAGTTTGCAACTTGTTG |
| | AGATTAAAGGTATGACTGACTTGTTCGAAGACGGGCTCCAAACGAT |
| | GCGCATGAATTACTATCCTCCATGTCCACGGCCTGAGTTGGTATTG |
| | GGTCTTACAAGTCATAGTGACTTTTCTGGGTTGACCATTTTACTCCA |
| | GTTAAACGAAGTGGAGGGGTTGCAGATTCGGAAGGAAGCACGGTG |
| | GATCAGCATCAAACCGCTTCCAGACGCCTTTATTGTCAACGTAGGT |
| | GATATTCTCGAAATTATGACCAACGGGATCTATCGTAGTGTTGAGCA |
| | CCGTGCAGTCGTGAATAGTACCAAGGAGCGGCTTTCCATCGCCACA |
| | TTCCATGACTCTAAATTGGAATCCGAAATCGGTCCTATCTCTTCGTT |
| | GGTTACTCCTGAGACCCCTGCATTATTCAAGCGCGGGCGCTACGAA |
| | GACATTCTCAAGGAAAACCTTTCGCGCAAACTTGATGGCAAGTCTTT |
| | CCTGGATTACATGCGCATGTGA |
| CODM | ATGGAGACCCCAATCTTGATCAAACTTGGCAACGGGCTCTCGATCC |
| E259S | CTAGCGTCCAAGAGCTCGCCAAGTTGACCCTGGCCGAGATCCCAA |
| SEQ ID | GTCGGTATACATGCACAGGTGAGAGCCCACTTAACAACATCGGTGC |
| NO: 7 | GTCAGTAACAGACGATGAAACGGTGCCGGTCATTGATTTGCAAAAC |
| | TTATTAAGTCCAGAGCCAGTAGTGGGGAAATTAGAGTTGGACAAGT |
| | TACACTCCGCTTGCAAAGAGTGGGGCTTCTTTCAGCTTGTCAACCA |
| | TGGCGTTGATGCCTTGTTAATGGACAACATTAAGAGCGAAATCAAG |
| | GGCTTTTTTAACCTGCCGATGAATGAGAAGACCAAATACGGTCAGC |
| | AGGACGGCGACTTTGAAGGGTTCGGTCAGCCTTATATTGAATCTGA |
| | GGATCAGCGCTTGGATTGGACTGAGGTGTTCTCAATGCTCTCGCTG |
| | CCACTTCACCTGCGCAAGCCGCACCTTTTTCCAGAACTTCCACTTC |
| | CGTTTCGCGAGACCCTGGAGTCGTACTTGAGCAAGATGAAAAAACT |
| | GTCAACGGTGGTGTTTGAGATGTTAGAAAAGAGTTTGCAACTTGTTG |
| | AGATTAAAGGTATGACTGACTTGTTCGAAGACGGGCTCCAAACGAT |
| | GCGCATGAATTACTATCCTCCATGTCCACGGCCTGAGTTGGTATTG |
| | GGTCTTACAAGTCATAGTGACTTTTCTGGGTTGACCATTTTACTCCA |
| | GTTAAACGAAGTGGAGGGGTTGCAGATTCGGAAGGAAAGTCGGTG |
| | GATCAGCATCAAACCGCTTCCAGACGCCTTTATTGTCAACGTAGGT |
| | GATATTCTCGAAATTATGACCAACGGGATCTATCGTAGTGTTGAGCA |
| | CCGTGCAGTCGTGAATAGTACCAAGGAGCGGCTTTCCATCGCCACA |
| | TTCCATGACTCTAAATTGGAATCCGAAATCGGTCCTATCTCTTCGTT |
| | GGTTACTCCTGAGACCCCTGCATTATTCAAGCGCGGGCGCTACGAA |
| | GACATTCTCAAGGAAAACCTTTCGCGCAAACTTGATGGCAAGTCTTT |
| | CCTGGATTACATGCGCATGTGA |
| CODM | ATGGAGACCCCAATCTTGATCAAACTTGGCAACGGGCTCTCGATCC |
| E259G | CTAGCGTCCAAGAGCTCGCCAAGTTGACCCTGGCCGAGATCCCAA |
| SEQ ID | GTCGGTATACATGCACAGGTGAGAGCCCACTTAACAACATCGGTGC |
| NO: 8 | GTCAGTAACAGACGATGAAACGGTGCCGGTCATTGATTTGCAAAAC |
| | TTATTAAGTCCAGAGCCAGTAGTGGGGAAATTAGAGTTGGACAAGT |
| | TACACTCCGCTTGCAAAGAGTGGGGCTTCTTTCAGCTTGTCAACCA |
| | TGGCGTTGATGCCTTGTTAATGGACAACATTAAGAGCGAAATCAAG |
| | GGCTTTTTTAACCTGCCGATGAATGAGAAGACCAAATACGGTCAGC |
| | AGGACGGCGACTTTGAAGGGTTCGGTCAGCCTTATATTGAATCTGA |
| | GGATCAGCGCTTGGATTGGACTGAGGTGTTCTCAATGCTCTCGCTG |
| | CCACTTCACCTGCGCAAGCCGCACCTTTTTCCAGAACTTCCACTTC |
| | CGTTTCGCGAGACCCTGGAGTCGTACTTGAGCAAGATGAAAAAACT |
| | GTCAACGGTGGTGTTTGAGATGTTAGAAAAGAGTTTGCAACTTGTTG |
| | AGATTAAAGGTATGACTGACTTGTTCGAAGACGGGCTCCAAACGAT |
| | GCGCATGAATTACTATCCTCCATGTCCACGGCCTGAGTTGGTATTG |
| | GGTCTTACAAGTCATAGTGACTTTTCTGGGTTGACCATTTTACTCCA |
| | GTTAAACGAAGTGGAGGGGTTGCAGATTCGGAAGGAAGGTCGGTG |
| | GATCAGCATCAAACCGCTTCCAGACGCCTTTATTGTCAACGTAGGT |
| | GATATTCTCGAAATTATGACCAACGGGATCTATCGTAGTGTTGAGCA |
| | CCGTGCAGTCGTGAATAGTACCAAGGAGCGGCTTTCCATCGCCACA |
| | TTCCATGACTCTAAATTGGAATCCGAAATCGGTCCTATCTCTTCGTT |
| | GGTTACTCCTGAGACCCCTGCATTATTCAAGCGCGGGCGCTACGAA |
| | GACATTCTCAAGGAAAACCTTTCGCGCAAACTTGATGGCAAGTCTTT |
| | CCTGGATTACATGCGCATGTGA |
| CODM | ATGGAGACCCCAATCTTGATCAAACTTGGCAACGGGCTCTCGATCC |
| R260T | CTAGCGTCCAAGAGCTCGCCAAGTTGACCCTGGCCGAGATCCCAA |
| SEQ ID | GTCGGTATACATGCACAGGTGAGAGCCCACTTAACAACATCGGTGC |
| NO: 9 | GTCAGTAACAGACGATGAAACGGTGCCGGTCATTGATTTGCAAAAC |
| | TTATTAAGTCCAGAGCCAGTAGTGGGGAAATTAGAGTTGGACAAGT |
| | TACACTCCGCTTGCAAAGAGTGGGGCTTCTTTCAGCTTGTCAACCA |
| | TGGCGTTGATGCCTTGTTAATGGACAACATTAAGAGCGAAATCAAG |
| | GGCTTTTTTAACCTGCCGATGAATGAGAAGACCAAATACGGTCAGC |
| | AGGACGGCGACTTTGAAGGGTTCGGTCAGCCTTATATTGAATCTGA |
| | GGATCAGCGCTTGGATTGGACTGAGGTGTTCTCAATGCTCTCGCTG |
| | CCACTTCACCTGCGCAAGCCGCACCTTTTTCCAGAACTTCCACTTC |
| | CGTTTCGCGAGACCCTGGAGTCGTACTTGAGCAAGATGAAAAAACT |
| | GTCAACGGTGGTGTTTGAGATGTTAGAAAAGAGTTTGCAACTTGTTG |
| | AGATTAAAGGTATGACTGACTTGTTCGAAGACGGGCTCCAAACGAT |
| | GCGCATGAATTACTATCCTCCATGTCCACGGCCTGAGTTGGTATTG |
| | GGTCTTACAAGTCATAGTGACTTTTCTGGGTTGACCATTTTACTCCA |
| | GTTAAACGAAGTGGAGGGGTTGCAGATTCGGAAGGAAGAGACGTG |
| | GATCAGCATCAAACCGCTTCCAGACGCCTTTATTGTCAACGTAGGT |
| | GATATTCTCGAAATTATGACCAACGGGATCTATCGTAGTGTTGAGCA |
| | CCGTGCAGTCGTGAATAGTACCAAGGAGCGGCTTTCCATCGCCACA |
| | TTCCATGACTCTAAATTGGAATCCGAAATCGGTCCTATCTCTTCGTT |
| | GGTTACTCCTGAGACCCCTGCATTATTCAAGCGCGGGCGCTACGAA |
| | GACATTCTCAAGGAAAACCTTTCGCGCAAACTTGATGGCAAGTCTTT |
| | CCTGGATTACATGCGCATGTGA |
| CODM | ATGGAGACCCCAATCTTGATCAAACTTGGCAACGGGCTCTCGATCC |
| E259G + | CTAGCGTCCAAGAGCTCGCCAAGTTGACCCTGGCCGAGATCCCAA |
| R260T | GTCGGTATACATGCACAGGTGAGAGCCCACTTAACAACATCGGTGC |
| SEQ ID | GTCAGTAACAGACGATGAAACGGTGCCGGTCATTGATTTGCAAAAC |
| NO: 10 | TTATTAAGTCCAGAGCCAGTAGTGGGGAAATTAGAGTTGGACAAGT |
| | TACACTCCGCTTGCAAAGAGTGGGGCTTCTTTCAGCTTGTCAACCA |
| | TGGCGTTGATGCCTTGTTAATGGACAACATTAAGAGCGAAATCAAG |
| | GGCTTTTTTAACCTGCCGATGAATGAGAAGACCAAATACGGTCAGC |
| | AGGACGGCGACTTTGAAGGGTTCGGTCAGCCTTATATTGAATCTGA |
| | GGATCAGCGCTTGGATTGGACTGAGGTGTTCTCAATGCTCTCGCTG |
| | CCACTTCACCTGCGCAAGCCGCACCTTTTTCCAGAACTTCCACTTC |
| | CGTTTCGCGAGACCCTGGAGTCGTACTTGAGCAAGATGAAAAAACT |
| | GTCAACGGTGGTGTTTGAGATGTTAGAAAAGAGTTTGCAACTTGTTG |
| | AGATTAAAGGTATGACTGACTTGTTCGAAGACGGGCTCCAAACGAT |
| | GCGCATGAATTACTATCCTCCATGTCCACGGCCTGAGTTGGTATTG |
| | GGTCTTACAAGTCATAGTGACTTTTCTGGGTTGACCATTTTACTCCA |
| | GTTAAACGAAGTGGAGGGGTTGCAGATTCGGAAGGAAGGTacgTGG |
| | ATCAGCATCAAACCGCTTCCAGACGCCTTTATTGTCAACGTAGGTG |
| | ATATTCTCGAAATTATGACCAACGGGATCTATCGTAGTGTTGAGCAC |
| | CGTGCAGTCGTGAATAGTACCAAGGAGCGGCTTTCCATCGCCACAT |
| | TCCATGACTCTAAATTGGAATCCGAAATCGGTCCTATCTCTTCGTTG |
| | GTTACTCCTGAGACCCCTGCATTATTCAAGCGCGGGCGCTACGAAG |
| | ACATTCTCAAGGAAAACCTTTCGCGCAAACTTGATGGCAAGTCTTTC |
| | CTGGATTACATGCGCATGTGA |
| CODM | CGGTCGTCAGACTGTCGATGAAGCCCTGAAAGACGCGCAGACTGG |
| R260K | ATCCATGGAGACCCCAATCTTGATCAAACTTGGCAACGGGCTCTCG |
| (UMg11) | ATCCCTAGCGTCCAAGAGCTCGCCAAGTTGACCCTGGCCGAGATC |
| SEQ ID | CCAAGTCGGTATACATGCACAGGTGAGAGCCCACTTAACAACATCG |
| NO: 11 | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGaa |
| | aTGGATCAGCATCAAACCGCTTCCAGACGCCTTTATTGTCAACGTAG |
| | GTGATATTCTCGAAATTATGACCAACGGGATCTATCGTAGTGTTGAG |
| | CACCGTGCAGTCGTGAATAGTACCAAGGAGCGGCTTTCCATCGCCA |
| | CATTCCATGACTCTAAATTGGAATCCGAAATCGGTCCTATCTCTTCG |
| | TTGGTTACTCCTGAGACCCCTGCATTATTCAAGCGCGGGCGCTACG |
| | AAGACATTCTCAAGGAAAACCTTTCGCGCAAACTTGATGGCAAGTCT |
| | TTCCTGGATTACATGCGCATGTGACTCGAGCACCACCACCACCACC |
| | ACTGAGATCCGGCTGCTAA |
| CODM | CGGTCGTCAGACTGTCGATGAAGCCCTGAAAGACGCGCAGACTGG |
| E259D + | ATCCATGGAGACCCCAATCTTGATCAAACTTGGCAACGGGCTCTCG |
| R260K | ATCCCTAGCGTCCAAGAGCTCGCCAAGTTGACCCTGGCCGAGATC |
| (UMg12) | CCAAGTCGGTATACATGCACAGGTGAGAGCCCACTTAACAACATCG |
| SEQ ID | GTGCGTCAGTAACAGACGATGAAACGGTGCCGGTCATTGATTTGCA |
| NO: 12 | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGACaa |
| | aTGGATCAGCATCAAACCGCTTCCAGACGCCTTTATTGTCAACGTAG |
| | GTGATATTCTCGAAATTATGACCAACGGGATCTATCGTAGTGTTGAG |
| | CACCGTGCAGTCGTGAATAGTACCAAGGAGCGGCTTTCCATCGCCA |
| | CATTCCATGACTCTAAATTGGAATCCGAAATCGGTCCTATCTCTTCG |
| | TTGGTTACTCCTGAGACCCCTGCATTATTCAAGCGCGGGCGCTACG |
| | AAGACATTCTCAAGGAAAACCTTTCGCGCAAACTTGATGGCAAGTCT |
| | TTCCTGGATTACATGCGCATGTGACTCGAGCACCACCACCACCACC |
| | ACTGAGATCCGGCTGCTAA |
| CODM | CGGTCGTCAGACTGTCGATGAAGCCCTGAAAGACGCGCAGACTGG |
| E259G + | ATCCATGGAGACCCCAATCTTGATCAAACTTGGCAACGGGCTCTCG |
| R260K | ATCCCTAGCGTCCAAGAGCTCGCCAAGTTGACCCTGGCCGAGATC |
| (UMg13) | CCAAGTCGGTATACATGCACAGGTGAGAGCCCACTTAACAACATCG |
| SEQ ID | GTGCGTCAGTAACAGACGATGAAACGGTGCCGGTCATTGATTTGCA |
| NO: 13 | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAggtaaa |
| | TGGATCAGCATCAAACCGCTTCCAGACGCCTTTATTGTCAACGTAG |
| | GTGATATTCTCGAAATTATGACCAACGGGATCTATCGTAGTGTTGAG |
| | CACCGTGCAGTCGTGAATAGTACCAAGGAGCGGCTTTCCATCGCCA |
| | CATTCCATGACTCTAAATTGGAATCCGAAATCGGTCCTATCTCTTCG |
| | TTGGTTACTCCTGAGACCCCTGCATTATTCAAGCGCGGGCGCTACG |
| | AAGACATTCTCAAGGAAAACCTTTCGCGCAAACTTGATGGCAAGTCT |
| | TTCCTGGATTACATGCGCATGTGACTCGAGCACCACCACCACCACC |
| | ACTGAGATCCGGCTGCTAA |
| CODM | CGGTCGTCAGACTGTCGATGAAGCCCTGAAAGACGCGCAGACTGG |
| WT* | ATCCATGGAAACCCCAATCCTGATCAAACTTGGCAACGGGCTCTCG |
| (UMg14) | ATCCCTAGCGTCCAAGAGCTCGCCAAGTTGACCCTGGCCGAGATC |
| SEQ ID | CCAAGTCGGTATACATGCACAGGTGAGAGCCCACTTAACAACATCG |
| NO: 37 | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATGTGACTCGAGCACCACCACCACC |
| | ACCACTGAGATCCGGCTGCTAA |
| CODM | CGGTCGTCAGACTGTCGATGAAGCCCTGAAAGACGCGCAGACTGG |
| T3K + P4A + | ATCCATGGAAAAAGCAAAACTGATGAAACTTGGCAACGGGCTCTCG |
| 15K + | ATCCCTAGCGTCCAAGAGCTCGCCAAGTTGACCCTGGCCGAGATC |
| 17M | CCAAGTCGGTATACATGCACAGGTGAGAGCCCACTTAACAACATCG |
| (UMg8) | GTGCGTCAGTAACAGACGATGAAACGGTGCCGGTCATTGATTTGCA |
| SEQ ID | AAACTTATTAAGTCCAGAGCCAGTAGTGGGGAAATTAGAGTTGGAC |
| NO: 38 | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGCATGTGACTCGAGCACCACCACCACC |
| | ACCACTGAGATCCGGCTGCTAA |
| CODM | CGGTCGTCAGACTGTCGATGAAGCCCTGAAAGACGCGCAGACTGG |
| Y357S + | ATCCATGGAGACCCCAATCTTGATCAAACTTGGCAACGGGCTCTCG |
| M3601 | ATCCCTAGCGTCCAAGAGCTCGCCAAGTTGACCCTGGCCGAGATC |
| (UMg9) | CCAAGTCGGTATACATGCACAGGTGAGAGCCCACTTAACAACATCG |
| SEQ ID | GTGCGTCAGTAACAGACGATGAAACGGTGCCGGTCATTGATTTGCA |
| NO: 39 | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTCTATGCGTATCTGACTCGAGCACCACCACCACCA |
| | CCACTGAGATCCGGCTGCTAA |
| CODM | CGGTCGTCAGACTGTCGATGAAGCCCTGAAAGACGCGCAGACTGG |
| T3K + P4A + | ATCCATGGAAAAAGCAAAACTGATGAAACTTGGCAACGGGCTCTCG |
| 15K + | ATCCCTAGCGTCCAAGAGCTCGCCAAGTTGACCCTGGCCGAGATC |
| 17M + | CCAAGTCGGTATACATGCACAGGTGAGAGCCCACTTAACAACATCG |
| Y357S + | GTGCGTCAGTAACAGACGATGAAACGGTGCCGGTCATTGATTTGCA |
| M3601 | AAACTTATTAAGTCCAGAGCCAGTAGTGGGGAAATTAGAGTTGGAC |
| (UMg10) | AAGTTACACTCCGCTTGCAAAGAGTGGGGCTTCTTTCAGCTTGTCA |
| SEQ ID | ACCATGGCGTTGATGCCTTGTTAATGGACAACATTAAGAGCGAAAT |
| NO: 40 | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTCTATGCGTATCTGACTCGAGCACCACCACCACCA |
| | CCACTGAGATCCGGCTGCTAA |
| CODM | CGGTCGTCAGACTGTCGATGAAGCCCTGAAAGACGCGCAGACTGG |
| T3K | ATCCATGGAAAAACCAATCCTGATCAAACTTGGCAACGGGCTCTCG |
| (UMg15) | ATCCCTAGCGTCCAAGAGCTCGCCAAGTTGACCCTGGCCGAGATC |
| SEQ ID | CCAAGTCGGTATACATGCACAGGTGAGAGCCCACTTAACAACATCG |
| NO: 41 | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATGTGACTCGAGCACCACCACCACC |
| | ACCACTGAGATCCGGCTGCTAA |
| CODM | CGGTCGTCAGACTGTCGATGAAGCCCTGAAAGACGCGCAGACTGG |
| P4A | ATCCATGGAAACCGCAATCCTGATCAAACTTGGCAACGGGCTCTCG |
| (UMg16) | ATCCCTAGCGTCCAAGAGCTCGCCAAGTTGACCCTGGCCGAGATC |
| SEQ ID | CCAAGTCGGTATACATGCACAGGTGAGAGCCCACTTAACAACATCG |
| NO: 42 | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATGTGACTCGAGCACCACCACCACC |
| | ACCACTGAGATCCGGCTGCTAA |
| CODM 15K | CGGTCGTCAGACTGTCGATGAAGCCCTGAAAGACGCGCAGACTGG |
| (UMg17) | ATCCATGGAAACCCCAAAACTGATCAAACTTGGCAACGGGCTCTCG |
| SEQ ID | ATCCCTAGCGTCCAAGAGCTCGCCAAGTTGACCCTGGCCGAGATC |
| NO: 43 | CCAAGTCGGTATACATGCACAGGTGAGAGCCCACTTAACAACATCG |
| | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATGTGACTCGAGCACCACCACCACC |
| | ACCACTGAGATCCGGCTGCTAA |
| CODM 17M | CGGTCGTCAGACTGTCGATGAAGCCCTGAAAGACGCGCAGACTGG |
| (UMg18) | ATCCATGGAAACCCCAATCCTGATGAAACTTGGCAACGGGCTCTCG |
| SEQ ID | ATCCCTAGCGTCCAAGAGCTCGCCAAGTTGACCCTGGCCGAGATC |
| NO: 44 | CCAAGTCGGTATACATGCACAGGTGAGAGCCCACTTAACAACATCG |
| | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATGTGACTCGAGCACCACCACCACC |
| | ACCACTGAGATCCGGCTGCTAA |
| CODM | CGGTCGTCAGACTGTCGATGAAGCCCTGAAAGACGCGCAGACTGG |
| Y357S | ATCCATGGAAACCCCAATCCTGATCAAACTTGGCAACGGGCTCTCG |
| (UMg19) | ATCCCTAGCGTCCAAGAGCTCGCCAAGTTGACCCTGGCCGAGATC |
| NO: 45 | CCAAGTCGGTATACATGCACAGGTGAGAGCCCACTTAACAACATCG |
| | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTCTATGCGTATGTGACTCGAGCACCACCACCACC |
| | ACCACTGAGATCCGGCTGCTAA |
| CODM | CGGTCGTCAGACTGTCGATGAAGCCCTGAAAGACGCGCAGACTGG |
| M3601 | ATCCATGGAAACCCCAATCCTGATCAAACTTGGCAACGGGCTCTCG |
| (UMg20) | ATCCCTAGCGTCCAAGAGCTCGCCAAGTTGACCCTGGCCGAGATC |
| SEQ ID | CCAAGTCGGTATACATGCACAGGTGAGAGCCCACTTAACAACATCG |
| NO: 46 | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATCTGACTCGAGCACCACCACCACC |
| | ACCACTGAGATCCGGCTGCTAA |
| CODM 15K + | CGGTCGTCAGACTGTCGATGAAGCCCTGAAAGACGCGCAGACTGG |
| M3601 | ATCCATGGAAACCCCAAAACTGATCAAACTTGGCAACGGGCTCTCG |
| (UMg21) | ATCCCTAGCGTCCAAGAGCTCGCCAAGTTGACCCTGGCCGAGATC |
| SEQ ID | CCAAGTCGGTATACATGCACAGGTGAGAGCCCACTTAACAACATCG |
| NO: 47 | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGAGC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATCTGACTCGAGCACCACCACCACC |
| | ACCACTGAGATCCGGCTGCTAA |
| CODM 15K + | CGGTCGTCAGACTGTCGATGAAGCCCTGAAAGACGCGCAGACTGG |
| E259G | ATCCATGGAAACCCCAAAACTGATCAAACTTGGCAACGGGCTCTCG |
| (UMg22) | ATCCCTAGCGTCCAAGAGCTCGCCAAGTTGACCCTGGCCGAGATC |
| SEQ ID | CCAAGTCGGTATACATGCACAGGTGAGAGCCCACTTAACAACATCG |
| NO: 48 | GTGCGTCAGTAACAGACGATGAAACGGTGCCGGTCATTGATTTGCA |
| | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGGTC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATGTGACTCGAGCACCACCACCACC |
| | ACCACTGAGATCCGGCTGCTAA |
| CODM | CGGTCGTCAGACTGTCGATGAAGCCCTGAAAGACGCGCAGACTGG |
| E259G + | ATCCATGGAAACCCCAATCCTGATCAAACTTGGCAACGGGCTCTCG |
| M3601 | ATCCCTAGCGTCCAAGAGCTCGCCAAGTTGACCCTGGCCGAGATC |
| (UMg23) | CCAAGTCGGTATACATGCACAGGTGAGAGCCCACTTAACAACATCG |
| SEQ ID | GTGCGTCAGTAACAGACGATGAAACGGTGCCGGTCATTGATTTGCA |
| NO: 49 | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGGTC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATCTGACTCGAGCACCACCACCACC |
| | ACCACTGAGATCCGGCTGCTAA |
| CODM 15K + | CGGTCGTCAGACTGTCGATGAAGCCCTGAAAGACGCGCAGACTGG |
| E259G + | ATCCATGGAAACCCCAAAACTGATCAAACTTGGCAACGGGCTCTCG |
| M3601 | ATCCCTAGCGTCCAAGAGCTCGCCAAGTTGACCCTGGCCGAGATC |
| (UMg24) | CCAAGTCGGTATACATGCACAGGTGAGAGCCCACTTAACAACATCG |
| SEQ ID | GTGCGTCAGTAACAGACGATGAAACGGTGCCGGTCATTGATTTGCA |
| NO: 50 | AAACTTATTAAGTCCAGAGCCAGTAGTGGGGAAATTAGAGTTGGAC |
| | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGGTC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATCTGACTCGAGCACCACCACCACC |
| | ACCACTGAGATCCGGCTGCTAA |
| CODM | CGGTCGTCAGACTGTCGATGAAGCCCTGAAAGACGCGCAGACTGG |
| P4A + | ATCCATGGAAACCGCAATCCTGATCAAACTTGGCAACGGGCTCTCG |
| E259G + | ATCCCTAGCGTCCAAGAGCTCGCCAAGTTGACCCTGGCCGAGATC |
| M3601 | CCAAGTCGGTATACATGCACAGGTGAGAGCCCACTTAACAACATCG |
| (UMg25) | GTGCGTCAGTAACAGACGATGAAACGGTGCCGGTCATTGATTTGCA |
| SEQ ID | AAACTTATTAAGTCCAGAGCCAGTAGTGGGGAAATTAGAGTTGGAC |
| NO: 51 | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGGTC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATCTGACTCGAGCACCACCACCACC |
| | ACCACTGAGATCCGGCTGCTAA |
| CODM | CGGTCGTCAGACTGTCGATGAAGCCCTGAAAGACGCGCAGACTGG |
| P4A + 15K + | ATCCATGGAAACCGCAAAACTGATCAAACTTGGCAACGGGCTCTCG |
| E259G + | ATCCCTAGCGTCCAAGAGCTCGCCAAGTTGACCCTGGCCGAGATC |
| M3601 | CCAAGTCGGTATACATGCACAGGTGAGAGCCCACTTAACAACATCG |
| (UMg26) | GTGCGTCAGTAACAGACGATGAAACGGTGCCGGTCATTGATTTGCA |
| SEQ ID | AAACTTATTAAGTCCAGAGCCAGTAGTGGGGAAATTAGAGTTGGAC |
| NO: 52 | AAGTTACACTCCGCTTGCAAAGAGTGGGGCTTCTTTCAGCTTGTCA |
| | ACCATGGCGTTGATGCCTTGTTAATGGACAACATTAAGAGCGAAAT |
| | CAAGGGCTTTTTTAACCTGCCGATGAATGAGAAGACCAAATACGGT |
| | CAGCAGGACGGCGACTTTGAAGGGTTCGGTCAGCCTTATATTGAAT |
| | CTGAGGATCAGCGCTTGGATTGGACTGAGGTGTTCTCAATGCTCTC |
| | GCTGCCACTTCACCTGCGCAAGCCGCACCTTTTTCCAGAACTTCCA |
| | CTTCCGTTTCGCGAGACCCTGGAGTCGTACTTGAGCAAGATGAAAA |
| | AACTGTCAACGGTGGTGTTTGAGATGTTAGAAAAGAGTTTGCAACTT |
| | GTTGAGATTAAAGGTATGACTGACTTGTTCGAAGACGGGCTCCAAA |
| | CGATGCGCATGAATTACTATCCTCCATGTCCACGGCCTGAGTTGGT |
| | ATTGGGTCTTACAAGTCATAGTGACTTTTCTGGGTTGACCATTTTAC |
| | TCCAGTTAAACGAAGTGGAGGGGTTGCAGATTCGGAAGGAAGGTC |
| | GGTGGATCAGCATCAAACCGCTTCCAGACGCCTTTATTGTCAACGT |
| | AGGTGATATTCTCGAAATTATGACCAACGGGATCTATCGTAGTGTTG |
| | AGCACCGTGCAGTCGTGAATAGTACCAAGGAGCGGCTTTCCATCGC |
| | CACATTCCATGACTCTAAATTGGAATCCGAAATCGGTCCTATCTCTT |
| | CGTTGGTTACTCCTGAGACCCCTGCATTATTCAAGCGCGGGCGCTA |
| | CGAAGACATTCTCAAGGAAAACCTTTCGCGCAAACTTGATGGCAAG |
| | TCTTTCCTGGATTACATGCGTATCTGACTCGAGCACCACCACCACC |
| | ACCACTGAGATCCGGCTGCTAA |

TABLE 4
P. somniferum Open Reading Frames

| Name | Sequence |
|---|---|
| CODM WT | ATGGAGACACCAATACTTATCAAGCTAGGCAATGG |
| (P. somniferum | TTTGTCAATACCAAGTGTTCAGGAATTGGCTAAAC |
| optimization) | TCACGCTTGCAGAAATTCCATCTCGATACACATGC |
| SEQ ID | ACCGGTGAAAGCCCGTTGAATAATATTGGTGCGTC |
| NO: 14 | TGTAACAGATGATGAAACAGTTCCTGTCATCGATT |
| | TGCAAAATTTACTATCTCCAGAACCCGTAGTTGGA |
| | AAGTTAGAATTGGATAAGCTTCATTCTGCTTGCAA |
| | AGAATGGGGTTTCTTTCAGCTGGTTAACCATGGAG |
| | TCGACGCTTTACTGATGGACAATATAAAATCAGAA |
| | ATTAAAGGTTTCTTTAACCTTCCAATGAATGAGAA |
| | AACTAAATACGGACAGCAAGATGGAGATTTTGAAG |
| | GATTTGGACAACCCTATATTGAATCGGAGGACCAA |
| | AGACTTGATTGGACTGAAGTGTTTAGCATGTTAAG |
| | TCTTCCTCTCCATTTAAGGAAGCCTCATTTGTTTC |
| | CAGAACTCCCTCTGCCTTTCAGGGAGACACTGGAA |
| | TCCTACCTATCAAAAATGAAAAAACTATCAACGGT |
| | TGTCTTTGAGATGTTGGAAAAATCTCTACAATTAG |
| | TTGAGATTAAAGGTATGACAGACTTATTTGAAGAT |
| | GGGTTGCAAACAATGAGGATGAACTATTATCCTCC |
| | TTGTCCTCGACCAGAGCTTGTACTTGGTCTTACGT |
| | CACACTCGGATTTTAGCGGTTTGACAATTCTCCTT |
| | CAACTTAATGAAGTTGAAGGATTACAAATAAGAAA |
| | AGAAGAGAGGTGGATTTCAATCAAACCTCTACCTG |
| | ATGCGTTCATAGTGAATGTTGGAGACATTTTGGAG |
| | ATAATGACTAATGGGATTTACCGTAGCGTCGAGCA |
| | CCGGGCAGTAGTAAACTCAACAAAGGAGAGGCTCT |
| | CAATCGCGACATTTCATGACTCTAAACTAGAGTCA |
| | GAAATAGGCCCAATTTCGAGCTTGGTCACACCAGA |
| | GACACCTGCTTTGTTCAAAAGAGGTAGGTATGAGG |
| | ATATTTTGAAGGAAAATCTTTCAAGGAAGCTTGAT |
| | GGAAAATCATTTCTCGACTACATGAGGATGTGA |
| CODM | ATGGAGACACCAATACTTATCAAGCTAGGCAATGG |
| E259K | TTTGTCAATACCAAGTGTTCAGGAATTGGCTAAAC |
| (P. somniferum | TCACGCTTGCAGAAATTCCATCTCGATACACATGC |
| optimization) | ACCGGTGAAAGCCCGTTGAATAATATTGGTGCGTC |
| SEQ ID | TGTAACAGATGATGAAACAGTTCCTGTCATCGATT |
| NO: 15 | TGCAAAATTTACTATCTCCAGAACCCGTAGTTGGA |
| | AAGTTAGAATTGGATAAGCTTCATTCTGCTTGCAA |
| | AGAATGGGGTTTCTTTCAGCTGGTTAACCATGGAG |
| | TCGACGCTTTACTGATGGACAATATAAAATCAGAA |
| | ATTAAAGGTTTCTTTAACCTTCCAATGAATGAGAA |
| | AACTAAATACGGACAGCAAGATGGAGATTTTGAAG |
| | GATTTGGACAACCCTATATTGAATCGGAGGACCAA |
| | AGACTTGATTGGACTGAAGTGTTTAGCATGTTAAG |
| | TCTTCCTCTCCATTTAAGGAAGCCTCATTTGTTTC |
| | CAGAACTCCCTCTGCCTTTCAGGGAGACACTGGAA |
| | TCCTACCTATCAAAAATGAAAAAACTATCAACGGT |
| | TGTCTTTGAGATGTTGGAAAAATCTCTACAATTAG |
| | TTGAGATTAAAGGTATGACAGACTTATTTGAAGAT |
| | GGGTTGCAAACAATGAGGATGAACTATTATCCTCC |
| | TTGTCCTCGACCAGAGCTTGTACTTGGTCTTACGT |
| | CACACTCGGATTTTAGCGGTTTGACAATTCTCCTT |
| | CAACTTAATGAAGTTGAAGGATTACAAATAAGAAA |
| | AGAGAAGAGGTGGATTTCAATCAAACCTCTACCTG |
| | ATGCGTTCATAGTGAATGTTGGAGACATTTTGGAG |
| | ATAATGACTAATGGGATTTACCGTAGCGTCGAGCA |
| | CCGGGCAGTAGTAAACTCAACAAAGGAGAGGCTCT |
| | CAATCGCGACATTTCATGACTCTAAACTAGAGTCA |
| | GAAATAGGCCCAATTTCGAGCTTGGTCACACCAGA |
| | GACACCTGCTTTGTTCAAAAGAGGTAGGTATGAGG |
| | ATATTTTGAAGGAAAATCTTTCAAGGAAGCTTGAT |
| | GGAAAATCATTTCTCGACTACATGAGGATGTGA |
| T60DM | ATGGAGAAAGCAAAACTTATGAAGCTAGGTAATGG |
| SEQ ID | TATGGAAATACCAAGTGTTCAAGAATTGGCTAAAC |
| NO: 16 | TCACGCTTGCCGAAATTCCATCTCGATACGTATGC |
| | GCCAATGAAAACCTTTTGTTGCCTATGGGTGCATC |
| | TGTCATAAATGATCATGAAACCATTCCTGTCATCG |
| | ATATAGAAAATTTATTATCTCCAGAACCAATAATC |
| | GGAAAGTTAGAATTAGATAGGCTTCATTTTGCTTG |
| | CAAAGAATGGGGTTTTTTTCAGGTAGTGAACCATG |
| | GAGTCGACGCTTCATTGGTGGATAGTGTAAAATCA |
| | GAAATTCAAGGTTTCTTTAACCTTTCTATGGATGA |
| | GAAAACTAAATATGAACAGGAAGATGGAGATGTGG |
| | AAGGATTTGGACAAGGCTTTATTGAATCAGAGGAC |
| | CAAACACTTGATTGGGCAGATATATTTATGATGTT |
| | CACTCTTCCACTCCATTTAAGGAAGCCTCACTTAT |
| | TTTCAAAACTCCCAGTGCCTCTCAGGGAGACAATC |
| | GAATCCTACTCATCAGAAATGAAAAAGTTATCCAT |
| | GGTTCTCTTTAATAAGATGGAAAAAGCTCTACAAG |
| | TACAAGCAGCCGAGATTAAGGGTATGTCAGAGGTG |
| | TTTATAGATGGGACACAAGCAATGAGGATGAACTA |
| | TTATCCCCCTTGTCCTCAACCAAATCTCGCCATCG |
| | GTCTTACGTCGCACTCGGATTTTGGCGGTTTGACA |
| | ATCCTCCTTCAAATCAACGAAGTGGAAGGATTACA |
| | GATAAAAAGAGAGGGGACATGGATTTCAGTCAAAC |
| | CTCTACCTAATGCGTTCGTAGTGAATGTTGGAGAT |
| | ATTTTGGAGATAATGACTAATGGAATTTACCATAG |
| | TGTCGATCACCGGGCAGTAGTAAACTCAACAAATG |
| | AGAGGCTCTCAATCGCAACATTTCATGACCCTAGT |
| | CTAGAGTCGGTAATAGGCCCAATATCAAGCTTGAT |
| | TACTCCAGAGACACCTGCTTTGTTTAAAAGTGGAT |
| | CTACATATGGGGATCTTGTGGAGGAATGTAAAACA |
| | AGGAAGCTCGATGGAAAATCATTTCTTGACTCCAT |
| | GAGGATTTGA |
| NISO | ATGGACTCAGTATCAGCTGCTCTAGTATTTCATAG |
| SEQ ID | TTCCATATACTTGTGTGCAATGGCTCATCATGGTG |
| NO: 17 | TTTCAGGTCTAGTTGGGAAAATTGTAACTGAATTG |
| | GAGGTGAATTGTAATGCCGACGAATTTTATAAGAT |
| | TTTGAAGCGCGATGAAGATGTTCCACGGGCAGTTT |
| | CTGATCTTTTCCCTCCCGTCAAAATTGCCAAAGGA |
| | GATGGACTTGTTTCTGGTTGTATCAAGGAATGGGA |
| | CTGTGTTCTTGATGGTAAGGCGATGAGCGGCAAGG |
| | AGGAAACAACACACAACGATGAAACGAGGACTTTG |
| | CGTCACCGTGAATTGGAAGGAGACTTGATGAAGGA |
| | TTACAAGAAGTTTGATTCCATAATTGAAGTTAATC |
| | CAAAACCAAATGGACATGGAAGCATTGTGACGTGG |
| | TCAATTGAGTATGAGAAAATGAACGAAGATTCTCC |
| | GGCTCCCTTTGCTTATCTAGCTTCCTTCCATCAGA |
| | ACGTTGTGGAAGTTGATTCTCACCTCTGCCTTTCT |
| | GAATAA |
| COR1-4 | ATGGAGAGTAATGGTGTACCTATGATCACTCTCAG |
| SEQ ID | TTCCGGCATTCGGATGCCTGCTTTAGGTATGGGAA |
| NO: 18 | CAGCTGAAACAATGGTAAAAGGAACAGAAAGAGAG |
| | AAATTGGCGTTTTTGAAAGCGATAGAGGTCGGTTA |
| | CAGACACTTCGATACAGCTGCTGCATACCAAAGTG |
| | AAGAGTGTCTTGGTGAAGCTATAGCTGAAGCACTT |
| | CAACTTGGTTTAATAAAATCTCGAGATGAACTCTT |
| | CATCACTTCCAAGCTCTGGTGCGCTGATGCTCACG |
| | CTGATCTTGTCCTCCCTGCTCTTCAGAATTCTCTG |
| | AGGAATCTCAAATTGGAGTATCTTGATCTATATTT |
| | GATACACCATCCGGTAAGCTTGAAGCCAGGGAAAT |
| | TTGTTAACGAAATACCAAAGGATCATATTCTTCCA |
| | ATGGACTACAAATCTGTATGGGCAGCCATGGAAGA |
| | GTGTCAGACCCTTGGCTTCACTAGGGCAATCGGTG |
| | TCAGTAATTTCTCATGCAAAAAGCTTCAAGAGTTG |
| | ATGGCAGCAGCCAAGATCCCTCCAGTTGTGAATCA |
| | AGTGGAGATGAGCCCTACTTTACATCAAAAAAATC |
| | TGAGGGAATATTGCAAGGCCAATAATATCATGATC |
| | ACTGCACACTCGGTTTTGGGAGCCATAGGTGCTCC |
| | ATGGGGCAGCAATGCAGTTATGGATTCTAAGGTGC |
| | TTCACCAGATTGCTGTGGCAAGAGGAAAATCTGTT |
| | GCCCAGGTTAGTATGAGATGGGTTTACCAGCAAGG |
| | CGCGAGTCTTGTGGTGAAAAGTTTCAATGAAGGGA |
| | GGATGAAGGAAAACCTTAAGATATTTGATTGGGAA |
| | CTAACGGCAGAAGATATGGAAAAGATCAGTGAGAT |
| | TCCGCAATCTAGAACAAGCTCTGCTGCTTTCTTGT |
| | TATCACCGACTGGACCTTTCAAAACTGAAGAAGAG |
| | TTCTGGGATGAGAAGGATTGA |

TABLE 5
Protein sequences

| Name | Sequence |
|---|---|
| CODM WT | METPILIKLGNGLSIPSVQELAKLTLAEIPSRYTCTGESPLNNIG |
| SEQ ID | ASVTDDETVPVIDLQNLLSPEPVVGKLELDKLHSACKEWGFFQLV |
| NO: 19 | NHGVDALLMDNIKSEIKGFFNLPMNEKTKYGQQDGDFEGFGQPYI |
| | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259K | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 20 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEKRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259D | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 21 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEDRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259H | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 22 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEHRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259Q | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 23 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEQRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259A | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 24 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEARWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259S | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 25 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKESRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259G | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 26 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEGRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| R260T | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 27 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEETWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259G + | ASVTDDETVPVIDLQNLLSPEPVVGKLELDKLHSACKEWGFFQLV |
| R260T | NHGVDALLMDNIKSEIKGFFNLPMNEKTKYGQQDGDFEGFGQPYI |
| SEQ ID | ESEDQRLDWTEVFSMLSLPLHLRKPHLFPELPLPFRETLESYLSK |
| NO: 28 | MKKLSTVVFEMLEKSLQLVEIKGMTDLFEDGLQTMRMNYYPPCPR |
| | PELVLGLTSHSDFSGLTILLQLNEVEGLQIRKEGTWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| R260K | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 29 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEEKWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259D + | ASVTDDETVPVIDLQNLLSPEPVVGKLELDKLHSACKEWGFFQLV |
| R260K | NHGVDALLMDNIKSEIKGFFNLPMNEKTKYGQQDGDFEGFGQPYI |
| SEQ ID | ESEDQRLDWTEVFSMLSLPLHLRKPHLFPELPLPFRETLESYLSK |
| NO: 30 | MKKLSTVVFEMLEKSLQLVEIKGMTDLFEDGLQTMRMNYYPPCPR |
| | PELVLGLTSHSDFSGLTILLQLNEVEGLQIRKEDKWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259G + | ASVTDDETVPVIDLQNLLSPEPVVGKLELDKLHSACKEWGFFQLV |
| R260K | NHGVDALLMDNIKSEIKGFFNLPMNEKTKYGQQDGDFEGFGQPYI |
| SEQ ID | ESEDQRLDWTEVFSMLSLPLHLRKPHLFPELPLPFRETLESYLSK |
| NO: 31 | MKKLSTVVFEMLEKSLQLVEIKGMTDLFEDGLQTMRMNYYPPCPR |
| | PELVLGLTSHSDFSGLTILLQLNEVEGLQIRKEGKWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| T60DM | MEKAKLMKLGNGMEIPSVQELAKLTLAEIPSRYVCANENLLLPMG |
| SEQ ID | ASVINDHETIPVIDIENLLSPEPIIGKLELDRLHFACKEWGFFQV |
| NO: 32 | VNHGVDASLVDSVKSEIQGFFNLSMDEKTKYEQEDGDVEGFGQGF |
| | IESEDQTLDWADIFMMFTLPLHLRKPHLFSKLPVPLRETIESYSS |
| | EMKKLSMVLFNKMEKALQVQAAEIKGMSEVFIDGTQAMRMNYYPP |
| | CPQPNLAIGLTSHSDFGGLTILLQINEVEGLQIKREGTWISVKPL |
| | PNAFVVNVGDILEIMTNGIYHSVDHRAVVNSTNERLSIATFHDPS |
| | LESVIGPISSLITPETPALFKSGSTYGDLVEECKTRKLDGKSFLD |
| | SMRI |
| NISO | MDSVSAALVFHSSIYLCAMAHHGVSGLVGKIVTELEVNCNADEFY |
| SEQ ID | KILKRDEDVPRAVSDLFPPVKIAKGDGLVSGCIKEWDCVLDGKAM |
| NO: 33 | SGKEETTHNDETRTLRHRELEGDLMKDYKKFDSIIEVNPKPNGHG |
| | SIVTWSIEYEKMNEDSPAPFAYLASFHQNVVEVDSHLCLSE |
| COR1-4 | MESNGVPMITLSSGIRMPALGMGTAETMVKGTEREKLAFLKAIEV |
| SEQ ID | GYRHFDTAAAYQSEECLGEAIAEALQLGLIKSRDELFITSKLWCA |
| NO: 34 | DAHADLVLPALQNSLRNLKLEYLDLYLIHHPVSLKPGKFVNEIPK |
| | DHILPMDYKSVWAAMEECQTLGFTRAIGVSNFSCKKLQELMAAAK |
| | IPPVVNQVEMSPTLHQKNLREYCKANNIMITAHSVLGAIGAPWGS |
| | NAVMDSKVLHQIAVARGKSVAQVSMRWVYQQGASLVVKSFNEGRM |
| | KENLKIFDWELTAEDMEKISEIPQSRTSSAAFLLSPTGPFKTEEE |
| | FWDEKD |
| CODM | METPILIKLGNGLSIPSVQELAKLTLAEIPSRYTCTGESPLNNIG |
| WT* | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 53 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | MEKAKLMKLGNGLSIPSVQELAKLTLAEIPSRYTCTGESPLNNIG |
| T3K + P4A | ASVTDDETVPVIDLQNLLSPEPVVGKLELDKLHSACKEWGFFQLV |
| + 15K + | NHGVDALLMDNIKSEIKGFFNLPMNEKTKYGQQDGDFEGFGQPYI |
| 17M | ESEDQRLDWTEVFSMLSLPLHLRKPHLFPELPLPFRETLESYLSK |
| SEQ ID | MKKLSTVVFEMLEKSLQLVEIKGMTDLFEDGLQTMRMNYYPPCPR |
| NO: 54 | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| Y357S + | ASVTDDETVPVIDLQNLLSPEPVVGKLELDKLHSACKEWGFFQLV |
| M3601 | NHGVDALLMDNIKSEIKGFFNLPMNEKTKYGQQDGDFEGFGQPYI |
| SEQ ID | ESEDQRLDWTEVFSMLSLPLHLRKPHLFPELPLPFRETLESYLSK |
| NO: 55 | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDSMRI |
| CODM | MEKAKLMKLGNGLSIPSVQELAKLTLAEIPSRYTCTGESPLNNIG |
| T3K + P4A | ASVTDDETVPVIDLQNLLSPEPVVGKLELDKLHSACKEWGFFQLV |
| + 15K + | NHGVDALLMDNIKSEIKGFFNLPMNEKTKYGQQDGDFEGFGQPYI |
| 17M + | ESEDQRLDWTEVFSMLSLPLHLRKPHLFPELPLPFRETLESYLSK |
| Y357S + | MKKLSTVVFEMLEKSLQLVEIKGMTDLFEDGLQTMRMNYYPPCPR |
| M3601 | PELVLGLTSHSDFSGLTILLQLNEVEGLQIRKEERWISIKPLPDA |
| SEQ ID | FIVNVGDILEIMTNGIYRSVEHRAVVNSTKERLSIATFHDSKLES |
| NO: 56 | EIGPISSLVTPETPALFKRGRYEDILKENLSRKLDGKSFLDSMRI |
| CODM | MEKPILIKLGNGLSIPSVQELAKLTLAEIPSRYTCTGESPLNNIG |
| T3K | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 57 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METAILIKLGNGLSIPSVQELAKLTLAEIPSRYTCTGESPLNNIG |
| P4A | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 58 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPKLIKLGNGLSIPSVQELAKLTLAEIPSRYTCTGESPLNNIG |
| 15K | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 59 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILMKLGNGLSIPSVQELAKLTLAEIPSRYTCTGESPLNNIG |
| 17M | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 60 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| Y357S | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 61 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDSMRM |
| CODM | METPILIKLGNGLSIPSVQELAKLTLAEIPSRYTCTGESPLNNIG |
| M3601 | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 62 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRI |
| CODM | METPKLIKLGNGLSIPSVQELAKLTLAEIPSRYTCTGESPLNNIG |
| 15K + | ASVTDDETVPVIDLQNLLSPEPVVGKLELDKLHSACKEWGFFQLV |
| M3601 | NHGVDALLMDNIKSEIKGFFNLPMNEKTKYGQQDGDFEGFGQPYI |
| SEQ ID | ESEDQRLDWTEVFSMLSLPLHLRKPHLFPELPLPFRETLESYLSK |
| NO: 63 | MKKLSTVVFEMLEKSLQLVEIKGMTDLFEDGLQTMRMNYYPPCPR |
| | PELVLGLTSHSDFSGLTILLQLNEVEGLQIRKEERWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRI |
| CODM 15K | METPKLIKLGNGLSIPSVQELAKLTLAEIPSRYTCTGESPLNNIG |
| + E259G | ASVTDDETVPVIDLQNLLSPEPVVGKLELDKLHSACKEWGFFQLV |
| SEQ ID | NHGVDALLMDNIKSEIKGFFNLPMNEKTKYGQQDGDFEGFGQPYI |
| NO: 64 | ESEDQRLDWTEVFSMLSLPLHLRKPHLFPELPLPFRETLESYLSK |
| | MKKLSTVVFEMLEKSLQLVEIKGMTDLFEDGLQTMRMNYYPPCPR |
| | PELVLGLTSHSDFSGLTILLQLNEVEGLQIRKEGRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRM |
| CODM | METPILIKLGNGLSIPSVQELAKLTLAEIPSRYTCTGESPLNNIG |
| E259G + | ASVTDDETVPVIDLQNLLSPEPVVGKLELDKLHSACKEWGFFQLV |
| M3601 | NHGVDALLMDNIKSEIKGFFNLPMNEKTKYGQQDGDFEGFGQPYI |
| SEQ ID | ESEDQRLDWTEVFSMLSLPLHLRKPHLFPELPLPFRETLESYLSK |
| NO: 65 | MKKLSTVVFEMLEKSLQLVEIKGMTDLFEDGLQTMRMNYYPPCPR |
| | PELVLGLTSHSDFSGLTILLQLNEVEGLQIRKEGRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRI |
| CODM 15K | METPKLIKLGNGLSIPSVQELAKLTLAEIPSRYTCTGESPLNNIG |
| + E259G + | ASVTDDETVPVIDLQNLLSPEPVVGKLELDKLHSACKEWGFFQLV |
| M3601 | NHGVDALLMDNIKSEIKGFFNLPMNEKTKYGQQDGDFEGFGQPYI |
| SEQ ID | ESEDQRLDWTEVFSMLSLPLHLRKPHLFPELPLPFRETLESYLSK |
| NO: 66 | MKKLSTVVFEMLEKSLQLVEIKGMTDLFEDGLQTMRMNYYPPCPR |
| | PELVLGLTSHSDFSGLTILLQLNEVEGLQIRKEGRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRI |
| CODM | METAILIKLGNGLSIPSVQELAKLTLAEIPSRYTCTGESPLNNIG |
| P4A + | ASVTDDETVPVIDLQNLLSPEPVVGKLELDKLHSACKEWGFFQLV |
| E259G + | NHGVDALLMDNIKSEIKGFFNLPMNEKTKYGQQDGDFEGFGQPYI |
| M3601 | ESEDQRLDWTEVFSMLSLPLHLRKPHLFPELPLPFRETLESYLSK |
| SEQ ID | MKKLSTVVFEMLEKSLQLVEIKGMTDLFEDGLQTMRMNYYPPCPR |
| NO: 67 | PELVLGLTSHSDFSGLTILLQLNEVEGLQIRKEGRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRI |
| CODM | METAKLIKLGNGLSIPSVQELAKLTLAEIPSRYTCTGESPLNNIG |
| P4A + 15K | ASVTDDETVPVIDLQNLLSPEPVVGKLELDKLHSACKEWGFFQLV |
| + E259G + | NHGVDALLMDNIKSEIKGFFNLPMNEKTKYGQQDGDFEGFGQPYI |
| M3601 | ESEDQRLDWTEVFSMLSLPLHLRKPHLFPELPLPFRETLESYLSK |
| SEQ ID | MKKLSTVVFEMLEKSLQLVEIKGMTDLFEDGLQTMRMNYYPPCPR |
| NO: 68 | PELVLGLTSHSDFSGLTILLQLNEVEGLQIRKEGRWISIKPLPDA |
| | FIVNVGDILEIMTNGIYRSVEHRAVVNSTKERLSIATFHDSKLES |
| | EIGPISSLVTPETPALFKRGRYEDILKENLSRKLDGKSFLDYMRI |

1. A method for the biotransformation of one or more opiate alkaloids or synthetic opioid alkaloids or derivatives thereof comprising the steps:
forming a preparation comprising a host cell transformed with one or more nucleic acid molecules encoding one or more polypeptides capable of catalysing at least one reaction in the conversion of one or more opiate alkaloids or synthetic opioid alkaloids or derivatives thereof to one or more different opiate alkaloids or semi-synthetic opioid alkaloids or derivatives thereof,
incubating the reaction to allow transformation of one or more opiate alkaloids or semi-synthetic opioid alkaloids or derivatives thereof; and optionally
extracting said one or more transformed different opiate alkaloids or semi-synthetic opioid alkaloids or derivatives thereof from said preparation.

2. The method according to claim 1 wherein, said nucleic acid molecule comprises a nucleotide sequence selected from the group consisting of:
i) a nucleotide sequence as set forth in SEQ ID NO: 1, 2, 3, 4, 5, 6, 7, 8, 9, 10, 11, 12, 13, 14, 15, 37, 38, 39, 40, 41, 42, 43, 44, 45, 46, 47, 48, 49, 50, 51 or 52;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridization conditions to the sequence set forth in SEQ ID NO: 1, 2, 3, 4, 5, 6, 7, 8, 9, 10, 11, 12, 13, 14, 15, 37, 38, 39, 40, 41, 42, 43, 44, 45, 46, 47, 48, 49, 50, 51 or 52 wherein said nucleic acid molecule encodes a codeine 3-O-demethylase;
iv) a nucleotide sequence that encodes a polypeptide comprising an amino acid sequence as represented in SEQ ID NO: 19, 20, 21, 22, 23, 24, 25, 26, 27, 28, 29, 30, 31, 53, 54, 55, 56, 57, 58, 59, 60, 61, 62, 63, 64, 65, 66, 67 or 68; and
v) a nucleotide sequence that encodes a polypeptide comprising an amino acid sequence wherein said amino acid sequence is modified by addition deletion or substitution of at least one amino acid residue as represented in iv) above and which has codeine 3-O-demethylase activity.

3. The method according to claim 1, wherein:
said nucleotide sequence encoding a codeine 3-O-demethylase polypeptide is set forth in SEQ ID NO: 3 or comprising a nucleotide sequence that is at least 90% identical to SEQ ID NO: 3;
said nucleotide sequence encoding a codeine 3-O-demethylase polypeptide is set forth in SEQ ID NO: 8 or comprising a nucleotide sequence that is at least 90% identical to SEQ ID NO: 8;
said polypeptide comprises an amino acid sequence as set forth in SEQ ID NO: 21, or a polypeptide comprising the amino acid sequence that is at least 90% identical to SEQ ID NO: 21 and comprises the amino acid substitution E259D;
said polypeptide comprises an amino acid sequence as set forth in SEQ ID NO: 21, or a polypeptide comprising the amino acid sequence that is at least 90% identical to SEQ ID NO: 21 and comprises the amino acid substitution E259D;
said nucleotide sequence encoding a codeine 3-O-demethylase polypeptide is set forth in SEQ ID NO: 63 or comprising a nucleotide sequence that is at least 90% identical to SEQ ID NO: 63;
said nucleotide sequence encoding a codeine 3-O-demethylase polypeptide is set forth in SEQ ID NO: 59 or comprising a nucleotide sequence that is at least 90% identical to SEQ ID NO: 59;
said nucleotide sequence encoding a codeine 3-O-demethylase polypeptide is set forth in SEQ ID NO: 68 or comprising a nucleotide sequence that is at least 90% identical to SEQ ID NO: 68;
said nucleotide sequence encoding a codeine 3-O-demethylase polypeptide is set forth in SEQ ID NO: 67 or comprising a nucleotide sequence that is at least 90% identical to SEQ ID NO: 67;
said nucleotide sequence encoding a codeine 3-O-demethylase polypeptide is set forth in SEQ ID NO: 66 or comprising a nucleotide sequence that is at least 90% identical to SEQ ID NO: 66;
said polypeptide comprises an amino acid sequence as set forth in SEQ ID NO: 63, or a polypeptide comprising the amino acid sequence that is at least 90% identical to SEQ ID NO: 63 and comprises the amino acid substitution 15K and M360I;
said polypeptide comprises an amino acid sequence as set forth in SEQ ID NO: 59, or a polypeptide comprising the amino acid sequence that is at least 90% identical to SEQ ID NO: 59 and comprises the amino acid substitution 15K;
said polypeptide comprises an amino acid sequence as set forth in SEQ ID NO: 68, or a polypeptide comprising the amino acid sequence that is at least 90% identical to SEQ ID NO: 68 and comprises the amino acid substitution P4A, 15K, E259G and M360I;
said polypeptide comprises an amino acid sequence as set forth in SEQ ID NO: 67, or a polypeptide comprising the amino acid sequence that is at least 90% identical to SEQ ID NO: 67 and comprises the amino acid substitution P4A, E259G and M360I; and/or
said polypeptide comprises an amino acid sequence as set forth in SEQ ID NO: 66, or a polypeptide comprising the amino acid sequence that is at least 90% identical to SEQ ID NO: 66 and comprises the amino acid substitution 15K, E259G and M360I.

4-17. (canceled)

18. The method according to claim 1, wherein said nucleic acid molecule comprises a nucleotide sequence selected from the group consisting of:
i) a nucleotide sequence as set forth in SEQ ID NO: 16;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridization conditions to the sequence in SEQ ID NO: 16 wherein said nucleic acid molecule encodes a thebaine 6-O-demethylase;
iv) a nucleotide sequence that encodes a polypeptide comprising an amino acid sequence as represented in SEQ ID NO: 32; and
v) a nucleotide sequence that encodes a polypeptide comprising an amino acid sequence wherein said amino acid sequence is modified by addition deletion or substitution of at least one amino acid residue as represented in iv) above and which has thebaine 6-O-demethylase activity.

19. The method according to claim 1, wherein said nucleic acid molecule comprises a nucleotide sequence selected from the group consisting of:
i) a nucleotide sequence as set forth in SEQ ID NO: 18;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridization conditions to the sequence in SEQ ID NO: 18 wherein said nucleic acid molecule encodes a codeinone reductase;
iv) a nucleotide sequence that encodes a polypeptide comprising an amino acid sequence as represented in SEQ ID NO: 34; and
v) a nucleotide sequence that encodes a polypeptide comprising an amino acid sequence wherein said amino acid sequence is modified by addition deletion or substitution of at least one amino acid residue as represented in iv) above and which has codeinone reductase activity.

20. The method according to claim 1 wherein said nucleic acid molecule comprises a nucleotide sequence selected from the group consisting of:
i) a nucleotide sequence as set forth in SEQ ID NO: 17;
ii) a nucleotide sequence wherein said sequence is degenerate as a result of the genetic code to the nucleotide sequence defined in (i);
iii) a nucleic acid molecule comprising a nucleotide sequence the complementary strand of which hybridizes under stringent hybridization conditions to the sequence in SEQ ID NO: 17 wherein said nucleic acid molecule encodes a neopinone isomerase;
iv) a nucleotide sequence that encodes a polypeptide comprising an amino acid sequence as represented in SEQ ID NO: 33; and
v) a nucleotide sequence that encodes a polypeptide comprising an amino acid sequence wherein said amino acid sequence is modified by addition deletion or substitution of at least one amino acid residue as represented in iv) above and which has neopinone isomerase activity.

21-22. (canceled)

23. The method according to claim 1, wherein said opiate alkaloids are obtained from crude poppy extract and obtained from a Papaver plant.

24. The method according to claim 23 wherein said Papaver plant is modified, wherein the plant is:
deleted or mutated for one, two or three linked codeine 3-O-demethylase genes encoded by a nucleic acid molecule comprising the nucleotide sequence set forth in SEQ ID NO: 1 or 37, or a nucleic acid molecule comprising a nucleotide sequence that is 95-99% identical to the nucleotide sequence set forth in SEQ ID NO: 1 or 37; and/or
deleted or mutated for one, two, three, four or five linked thebaine 6-O-demethylase genes encoded by a nucleic acid molecule comprising the nucleotide sequence set forth in SEQ ID NO: 16, or a nucleic acid molecule comprising a nucleotide sequence that is 95-99% identical to the nucleotide sequence set forth in SEQ ID NO: 16.

25. The method according to claim 24 wherein:
said Papaver plant is deleted for each codeine 3-O-demethylase gene wherein codeine 3-O-demethylase activity is undetectable; and/or
said Papaver plant is deleted for each thebaine 6-O-demethylase gene wherein thebaine 6-O-demethylase activity is undetectable.

26-27. (canceled)

28. The method according to claim 24 wherein said Papaver plant is modified and comprises:
a genomic modification to one, two or three genes encoding codeine 3-O-demethylases,
a genomic modification to one, two, three, four or five genes encoding thebaine 6-O-demethylases,
wherein the expression of codeine 3-O-demethylases or the activity of codeine 3-O-demethylases is reduced or undetectable and further wherein the expression of said thebaine 6-O-demethylases or activity of thebaine 6-O-demethylases is reduced or undetectable wherein the modified plant has elevated levels of thebaine when compared to a wild type Papaver somniferum plant and comprising functional genes encoding codeine 3-O-demethylases and functional genes encoding thebaine 6-O-demethylase(s).

29. A modified codeine 3-O-demethylase polypeptide wherein the activity of said modified codeine 3-O-demethylase polypeptide is altered when compared to a non-modified codeine 3-O-demethylase.

30. The modified codeine 3-O-demethylase polypeptide according to claim 29 wherein:
said modification is to an amino acid sequence as set forth in SEQ ID NO: 19 wherein said polypeptide is modified by addition, deletion or substitution of at least one amino acid residue wherein said codeine 3-O-demethylase has altered activity;
said modified codeine 3-O-demethylase polypeptide is modified at amino acid position 259 and/or amino acid position 260 or comprising an amino acid sequence that is at least 90% identical to SEQ ID NO: 19 and comprises a modification to amino acid residue 259 and/or 260;
said modification is to an amino acid sequence as set forth in SEQ ID NO: 53 wherein said polypeptide is modified by addition, deletion or substitution of at least one amino acid residue wherein said codeine 3-O-demethylase has altered activity;
said modified codeine 3-O-demethylase polypeptide is modified at one or more amino acid positions selected from 3, 4, 5, 7, 259, 357 and/or 360, or combinations thereof, or comprising an amino acid sequence that is at least 90% identical to SEQ ID NO: 53 and comprises a modification to a codon for one or more amino acid residue positions selected from 3, 4, 5, 7, 259, 357 and/or 360, or combinations thereof; and/or
said modified codeine 3-O-demethylase polypeptide has enhanced activity when compared to a wild-type codeine 3-O-demethylase polypeptide as represented by the amino acid sequence set forth in SEQ ID NO: 19 or 53.

31-33. (canceled)

34. The modified codeine 3-O-demethylase polypeptide according to claim 29, wherein said modified codeine 3-O-demethylase polypeptide is selected from SEQ ID NO: 20, 21, 22, 23, 24, 25, 26, 27, 28, 29, 30, 31, 54, 55, 56, 57, 58, 59, 60, 61, 62, 63, 64, 65, 66, 67 or 68.

35-36. (canceled)

37. The codeine 3-O-demethylase polypeptide according to claim 29, wherein:
said modified codeine 3-O-demethylase polypeptide is represented by the amino acid sequence set forth in SEQ ID NO: 21;
said modified codeine 3-O-demethylase polypeptide is represented by the amino acid sequence set forth in SEQ ID NO: 26;
said modified codeine 3-O-demethylase polypeptide is represented by the amino acid sequence set forth in SEQ ID NO: 63;
said modified codeine 3-O-demethylase polypeptide is represented by the amino acid sequence set forth in SEQ ID NO: 59;
said modified codeine 3-O-demethylase polypeptide is represented by the amino acid sequence set forth in SEQ ID NO: 68;
said modified codeine 3-O-demethylase polypeptide is represented by the amino acid sequence set forth in SEQ ID NO: 67; or
said modified codeine 3-O-demethylase polypeptide is represented by the amino acid sequence set forth in SEQ ID NO: 66.

38-43. (canceled)

44. An isolated nucleic acid molecule comprising a nucleotide sequence encoding a modified codeine 3-O-demethylase polypeptide according to claim 29.

45. The isolated nucleic acid molecule according to claim 44 wherein said nucleic acid molecule comprises a nucleotide sequence as set forth in SEQ ID NO: 2, 3, 4, 5, 6, 7, 8, 9, 10, 11, 12, 13, 14 or 15 or comprising a nucleotide sequence that is at least 90% identical to SEQ ID NO: 2, 3, 4, 5, 6, 7, 8, 9, 10, 11, 12, 13, 14 or 15 and has modified codeine 3-O-demethylase activity.

46. The isolated nucleic acid molecule according to claim 45 wherein said nucleotide sequence encoding a modified codeine 3-O-demethylase polypeptide is set forth in SEQ ID NO: 8 or comprising a nucleotide sequence that is at least 90% identical to SEQ ID NO: 8 and comprises a modification to a codon for amino acid residue 259 and optionally amino acid residue 260.

47. The isolated nucleic acid molecule according to claim 46 wherein:
said nucleotide sequence encodes a modified codeine 3-O-demethylase polypeptide comprising an amino acid sequence as set forth in SEQ ID NO: 21, or a polypeptide comprising the amino acid sequence that is at least 90% identical to SEQ ID NO: 21 and comprises the amino acid substitution E259D; and/or
said nucleotide sequence encodes a modified codeine 3-O-demethylase polypeptide comprising an amino acid sequence as set forth in SEQ ID NO: 26 or a polypeptide comprising the amino acid sequence that is at least 90% identical to SEQ ID NO: 26 and comprises the amino acid substitution E259G.

48. (canceled)

49. The isolated nucleic acid molecule according to claim 44 wherein said nucleic acid molecule comprises a nucleotide sequence as set forth in SEQ ID NO: 38, 39, 40, 41, 42, 43, 44, 45, 46, 47, 48, 49, 50, 51 or 52 or comprising a nucleotide sequence that is at least 90% identical to SEQ ID NO: 38, 39, 40, 41, 42, 43, 44, 45, 46, 47, 48, 49, 50, 51 or 52 and has modified codeine 3-O-demethylase activity.

50. The isolated nucleic acid molecule according to claim 49 wherein:
said nucleotide sequence encoding a modified codeine 3-O-demethylase polypeptide is set forth in SEQ ID NO: 43 or comprising a nucleotide sequence that is at least 90% identical to SEQ ID NO: 43 and comprises one or more modifications to a codon for amino acid residue 5 and optionally amino acid residue 360, 259, 3, 7, 357 and/or 4;
said nucleotide sequence encoding a modified codeine 3-O-demethylase polypeptide is set forth in SEQ ID NO: 51 or comprising a nucleotide sequence that is at least 90% identical to SEQ ID NO: 51 and comprises one or more modifications to a codon for amino acid residue 4, 360, 259 and optionally amino acid residue 3, 7, 357 and/or 5;
said nucleotide sequence encodes a modified codeine 3-O-demethylase polypeptide comprising an amino acid sequence as set forth in SEQ ID NO: 59, or a polypeptide comprising the amino acid sequence that is at least 90% identical to SEQ ID NO: 59 and comprises the amino acid substitution 15K;
said nucleotide sequence encodes a modified codeine 3-O-demethylase polypeptide comprising an amino acid sequence as set forth in SEQ ID NO: 63 or a polypeptide comprising the amino acid sequence that is at least 90% identical to SEQ ID NO: 63 and comprises the amino acid substitution 15K and M360I;
said nucleotide sequence encodes a modified codeine 3-O-demethylase polypeptide comprising an amino acid sequence as set forth in SEQ ID NO: 66 or a polypeptide comprising the amino acid sequence that is at least 90% identical to SEQ ID NO: 66 and comprises the amino acid substitution 15K, E259G and M360I;
said nucleotide sequence encodes a modified codeine 3-O-demethylase polypeptide comprising an amino acid sequence as set forth in SEQ ID NO: 67 or a polypeptide comprising the amino acid sequence that is at least 90% identical to SEQ ID NO: 67 and comprises the amino acid substitution P4A, E259G and M360I; and/or
said nucleotide sequence encodes a modified codeine 3-O-demethylase polypeptide comprising an amino acid sequence as set forth in SEQ ID NO: 68 or a polypeptide comprising the amino acid sequence that is at least 90% identical to SEQ ID NO: 68 and comprises the amino acid substitution P4A, 15K, E259G and M360I.

51-56. (canceled)

57. An isolated nucleic acid molecule comprising a nucleotide sequence encoding a modified codeine 3-O-demethylase polypeptide, wherein said nucleic acid molecule comprises a nucleotide sequence wherein said nucleotide sequence is degenerate because of the genetic code and encodes a modified codeine 3-O-demethylase polypeptide according to claim 29.

58. An expression vector comprising a nucleic acid molecule according to claim 44.

59. A cell transformed with a nucleic acid molecule according to claim 44 or a vector comprising the nucleic acid molecule.

60. A process for the biotransformation of a first opiate, opioid alkaloid, or semi-synthetic opiate alkaloid to a second opiate, opioid alkaloid, or semi-synthetic opiate alkaloid comprising:
forming a preparation comprising a modified codeine 3-O-demethylase polypeptide according to claim 29 and a crude poppy extract or a purified or semi-purified source of selected opiate or opioid alkaloid; and
incubating the preparation to allow transformation of one or more opiate or opioid alkaloids; and optionally extracting said one or more transformed opiate or opioid alkaloids from said preparation.

61. The process according to claim 60 wherein said modified codeine 3-O-demethylase polypeptide is expressed by a cell transformed with a nucleic acid molecule encoding the modified codeine 3-O-demethylase polypeptide or a vector comprising the nucleic acid molecule.

62. The process according to claim 60, wherein:
said first opiate alkaloid is thebaine and said second opiate alkaloid is oripavine;
said first opiate alkaloid is codeine and said second opiate alkaloid is morphine;
said first opiate alkaloid is codeinone and said second opiate alkaloid is morphinone;
said first semi-synthetic opioid alkaloid is oxycodone and said second semi-synthetic opioid alkaloid is oxymorphone;
said first semi-synthetic opioid alkaloid is hydrocodone and said second semi-synthetic opioid alkaloid is hydromorphone;
said first semi-synthetic opioid alkaloid is dihydrocodeine and said second semi-synthetic opioid alkaloid is dihydromorphine;
said first semi-synthetic opioid alkaloid is 14-hydroxycodeine and said second semi-synthetic opioid alkaloid is 14-hydroxymorphine;
said first semi-synthetic opioid alkaloid is noroxycodone and said second semi-synthetic opioid alkaloid is noroxymorphone;
said first semi-synthetic opioid alkaloid is noroxycodeinone and said second semi-synthetic opioid alkaloid is noroxymorphinone;
said first semi-synthetic opioid alkaloid is 14-hydroxy norcodeinone and said second semi-synthetic opioid alkaloid is 14-hydroxy normorphinone;
said first semi-synthetic opioid alkaloid is 14-hydroxycodeinone and said second semi-synthetic opioid alkaloid is 14-hydroxymorphinone;
said first semi-synthetic opioid alkaloid is buprenorphine intermediate (N-cyclopropylmethyl-7α-(2-hydroxy-3,3-dimethyl-2-butyl)-6,14-endoethano-6,7,8,14-tetrahydronorthebaine) and said second semi-synthetic opioid alkaloid is buprenorphine ((2S)-2-[17-(cyclopropylmethyl)-4,5α-epoxy-3-hydroxy-6-methoxy-6α,14-ethano-14α-morphinan-7α-γl]-3,3-dimethylbutan-2-ol); or
said first semi-synthetic opioid alkaloid is buprenorphine intermediate (7-acetyl-6,14-endoetheno-6,7,8,14-tetrahydrothebaine) and said second semi-synthetic opioid alkaloid is etorphine intermediate.

63-74. (canceled)
